SEQUENCE LISTING (30 sequences)

SEQ ID NO: 1 - protein, 13 aa, synthetic construct
TAFGGSISGHYWS

SEQ ID NO: 2 - protein, 9 aa, synthetic construct
RIYPSGGTN

SEQ ID NO: 3 - protein, 13 aa, synthetic construct
VRDYWDDYYGMDV

SEQ ID NO: 4 - protein, 16 aa, synthetic construct
RSSQSLLHSDGFNYLD

SEQ ID NO: 5 - protein, 8 aa, synthetic construct
YLGSNRAS

SEQ ID NO: 6 - protein, 9 aa, synthetic construct
MQAYQTPRT

SEQ ID NO: 7 - protein, 119 aa, synthetic construct
QVQLQESGPGLVKPSETLSLTCTAFGGSISGHYWSWIRQPAGKGLEWIGRIYPSGGTNKNPSLKSRVTMSVDTSKNQFSLKLSSVTAADTAVYYCVRDYWDDYYGMDVWGQGTTVTVSS

SEQ ID NO: 8 - protein, 112 aa, synthetic construct
DIVMTQSPLSLPVTPGEPASISCRSSQSLLHSDGFNYLDWYLQKPGQSPQLLIYLGSNRASGVPDRFSGSGSGTDFTLKISRVEAEDVGVYYCMQAYQTPRTFGQGTKVEIK

SEQ ID NO: 9 - protein, 444 aa, synthetic construct
QVQLQESGPGLVKPSETLSLTCTAFGGSISGHYWSWIRQPAGKGLEWIGRIYPSGGTNKNPSLKSRVTMSVDTSKNQFSLKLSSVTAADTAVYYCVRDYWDDYYGMDVWGQGTTVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 10 - protein, 219 aa, synthetic construct
DIVMTQSPLSLPVTPGEPASISCRSSQSLLHSDGFNYLDWYLQKPGQSPQLLIYLGSNRASGVPDRFSGSGSGTDFTLKISRVEAEDVGVYYCMQAYQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1335 bp, synthetic construct
  source 1..1335: mol_type = other DNA
caggtgcagctgcaggagtcgggcccaggactggtgaagccttcggagaccctgtccctcacctgtactgcctttggtggctccatcagtggtcactactggagctggatccggcagcccgccgggaagggactggagtggattgggcgtatttatcctagtgggggcaccaacaaaaacccctccctcaagagtcgcgtcaccatgtcagtagacacgtccaagaatcagttctccctgaagctgagttctgtgaccgccgcggacacggccgtgtattattgtgtgagagattactgggacgactactatggtatggacgtctggggccaagggaccacggtcaccgtctcctcagcctccaccaagggcccatcggtcttccccctggcgcccagctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggttga

SEQ ID NO: 12 - DNA, 657 bp, synthetic construct
  source 1..657: mol_type = other DNA
gatattgtgatgactcagtctccactctccctgcccgtcacccctggagagccggcctccatctcctgcaggtctagtcagagcctcctgcatagtgatggattcaactatttggattggtacctgcagaagccagggcagtctccacagctcctgatctatctgggttctaatcgggcctccggggtccctgacaggttcagtggcagtggatcaggcacagattttacactgaaaatcagcagagtggaggctgaggatgttggggtttattactgcatgcaagcttatcaaactcctcggacgttcggccaagggaccaaggtggaaatcaaacgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 13 - protein, 13 aa, synthetic construct
TVSGGSISNNYWS

SEQ ID NO: 14 - protein, 9 aa, synthetic construct
RMYSSGSTQ

SEQ ID NO: 15 - protein, 13 aa, synthetic construct
GPNWGSPYYGVDV

SEQ ID NO: 16 - protein, 16 aa, synthetic construct
RSSPSLLHSDGYNWLD

SEQ ID NO: 17 - protein, 148 aa, Homo sapiens
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHENLYFQ

SEQ ID NO: 18 - protein, 9 aa, synthetic construct
MQAHQTPFT

SEQ ID NO: 19 - protein, 121 aa, synthetic construct
QVQLQESGPGLVKPSETLSLTCTVSGGSISNNYWSWIRQPAGKGLEWIGRMYSSGSTQKNPSLKSRVTMSVDTSKNQFSLKLSSVTAADTAVYYCVRGPNWGSPYYGVDVWGQGTTVTVSS

SEQ ID NO: 20 - protein, 112 aa, synthetic construct
DIVMTQSPLSLPVTPGEPASISCRSSPSLLHSDGYNWLDWYLQKPGQSPQLLIYLGSNRASGVPDRFSGSGSGTDFTLKISRVEAEDVGVYYCMQAHQTPFTFGQGTKVEIK

SEQ ID NO: 21 - protein, 446 aa, synthetic construct
QVQLQESGPGLVKPSETLSLTCTVSGGSISNNYWSWIRQPAGKGLEWIGRMYSSGSTQKNPSLKSRVTMSVDTSKNQFSLKLSSVTAADTAVYYCVRGPNWGSPYYGVDVWGQGTTVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 22 - protein, 219 aa, synthetic construct
DIVMTQSPLSLPVTPGEPASISCRSSPSLLHSDGYNWLDWYLQKPGQSPQLLIYLGSNRASGVPDRFSGSGSGTDFTLKISRVEAEDVGVYYCMQAHQTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1341 bp, synthetic construct
  source 1..1341: mol_type = other DNA
caggtgcagctgcaggagtcgggcccaggactggtgaagccttcggagaccctgtccctcacctgcactgtctctggtggctccatcagtaataactactggagttggatccggcagcccgccggtaaggggctggagtggattgggcgtatgtattccagtgggagcacccagaaaaacccctccctcaagagtcgagtcaccatgtcagtagacacgtccaaaaaccagttctccctgaagctgtcctctgtgaccgccgcggacacggccgtgtattattgtgtgagaggccctaactggggatccccctactacggtgtggacgtctggggccaagggaccacggtcaccgtctcctcagcctccaccaagggcccatcggtcttccccctggcgcccagctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggttga

SEQ ID NO: 24 - DNA, 657 bp, synthetic construct
  source 1..657: mol_type = other DNA
gatattgtgatgactcagtctccactctccctgcccgtcacccctggagagccggcctccatctcctgcaggtctagtccgagcctcctgcatagtgatggatacaactggttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctcaccaaactccattcactttcggccaggggaccaaggtggaaatcaaacgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 25 - protein, 445 aa, synthetic construct
QVQLQESGPGLVKPSETLSLTCTAFGGSISGHYWSWIRQPAGKGLEWIGRIYPSGGTNKNPSLKSRVTMSVDTSKNQFSLKLSSVTAADTAVYYCVRDYWDDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - DNA, 1335 bp, synthetic construct
  source 1..1335: mol_type = other DNA
caggtgcagctgcaggagtcgggcccaggactggtgaagccttcggagaccctgtccctcacctgtactgcctttggtggctccatcagtggtcactactggagctggatccggcagcccgccgggaagggactggagtggattgggcgtatttatcctagtgggggcaccaacaaaaacccctccctcaagagtcgcgtcaccatgtcagtagacacgtccaagaatcagttctccctgaagctgagttctgtgaccgccgcggacacggccgtgtattattgtgtgagagattactgggacgactactatggtatggacgtctggggccaagggaccacggtcaccgtctcctcagctagcaccaagggcccatcggtcttcccactagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagtttctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 27 - protein, 447 aa, synthetic construct
QVQLQESGPGLVKPSETLSLTCTVSGGSISNNYWSWIRQPAGKGLEWIGRMYSSGSTQKNPSLKSRVTMSVDTSKNQFSLKLSSVTAADTAVYYCVRGPNWGSPYYGVDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - DNA, 1341 bp, synthetic construct
  source 1..1341: mol_type = other DNA
caggtgcagctgcaggagtcgggcccaggactggtgaagccttcggagaccctgtccctcacctgcactgtctctggtggctccatcagtaataactactggagttggatccggcagcccgccggtaaggggctggagtggattgggcgtatgtattccagtgggagcacccagaaaaacccctccctcaagagtcgagtcaccatgtcagtagacacgtccaaaaaccagttctccctgaagctgtcctctgtgaccgccgcggacacggccgtgtattattgtgtgagaggccctaactggggatccccctactacggtgtggacgtctggggccaagggaccacggtcaccgtctcctcagctagcaccaagggcccatcggtcttcccactagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagtttctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 29 - protein, 287 aa, Homo sapiens
MSPHPTALLGLVLCLAQTIHTQEEDLPRPSISAEPGTVIPLGSHVTFVCRGPVGVQTFRLERDSRSTYNDTEDVSQASPSESEARFRIDSVREGNAGLYRCIYYKPPKWSEQSDYLELLVKESSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 30 - protein, 387 aa, synthetic construct
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHENLYFQGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

(19) United States
(12) Patent Application Publication
(10) Pub. No.: US 2024/0101670 A1
(43) Pub. Date: Mar. 28, 2024

(54) ANTI-HUMAN LAIR1 ANTIBODIES

(71) Applicants: Eli Lilly and Company, Indianapolis, IN (US); TRex Bio, Inc., South San Francisco, CA (US)

(72) Inventors: Julian DAVIES, La Jolla, CA (US); Scott Charles POTTER, San Diego, CA (US); Andrew Charles VENDEL, San Diego, CA (US); Wei WANG, Zionsville, IN (US); Reid Martin Renny FELDMAN, San Francisco, CA (US); Shireen Syrah KHAN, Castro Valley, CA (US); Monica MACAL, San Bruno, CA (US); Maria Elena AYALA RAMIREZ, San Francisco, CA (US)

(21) Appl. No.: 18/467,957

(22) Filed: Sep. 15, 2023

Related U.S. Application Data
(60) Provisional application No. 63/375,936, filed on Sep. 16, 2022.

(51) Int. Cl. C07K 16/28; A61P 37/06

(52) U.S. Cl. CPC C07K 16/2803; A61P 37/06; A61K 2039/505

(57) ABSTRACT

The present disclosure relates to antibodies that bind human LAIR1 (“anti-human LAIR1 antibodies” or “anti-human LAIR1 antibodies”), compositions comprising such anti-human LAIR1 antibodies, and methods of using such anti-human LAIR1 antibodies.

### SEQUENCE LISTING FILE

The present application is being filed along with a Sequence Listing in ST.26 XML format. The Sequence Listing is provided as a file titled “30426 Sequence Listing” created 10 Aug. 2023 and is 38 kilobytes in size. The Sequence Listing information in the ST.26 XML format is incorporated herein by reference in its entirety.

### FIELD OF THE INVENTION

The present disclosure relates to antibodies that bind human LAIR1 (“anti-human LAIR1 antibodies” or “anti-LAIR1 antibodies” or “human LAIR1 antibodies”), compositions comprising such anti-human LAIR1 antibodies, and methods of using such anti-human LAIR1 antibodies.

### BACKGROUND

Human leukocyte associated immunoglobulin like receptor 1 (LAIR1, also known as CD305) is an inhibitory receptor found on peripheral mononuclear cells, including natural killer cells, T cells, B cells, macrophages, dendritic cells, as well as hematopoietic progenitors including human CD34+ cells. It belongs to the immunoglobulin superfamily and plays a role in regulating immune responses. Inhibitory receptors regulate the immune response to prevent lysis of cells recognized as self.

Structurally, LAIR1 is a type I transmembrane glycoprotein that contains an extracellular C2-type immunoglobulin-like domain, a stalk region, a single transmembrane domain, and an intracellular domain comprising two conserved motifs termed immunoreceptor tyrosine-based inhibitory motifs (ITIMs). LAIR1 is structurally related to several other inhibitory immunoglobulin superfamily members, including LILRBs, localized to the leukocyte receptor complex (LRC) on human chromosome 19q13.4, suggesting that these molecules have evolved from a common ancestral gene.

LAIR1 expression is altered in several autoimmune diseases such as systemic lupus erythematosus (SLE) and rheumatoid arthritis (RA) (see Zhang Y. et al., Clin Exp Immunol. 2018 May; 192(2):193-205). Due to the immune inhibitory function of LAIR1, there is a need for antibodies that modulate the activity of LAIR1, which can be used as therapeutics for treating autoimmune disease. Such antibodies may be used to treat autoimmune diseases, including SLE and lupus nephritis. The standard of care currently includes numerous steroids, which have many unfavourable and/or potentially dangerous side effects. There is, therefore, a need to find a safe and efficacious therapeutic treatment for such autoimmune diseases.

### SUMMARY OF INVENTION

Provided herein are novel anti-human LAIR1 antibodies or antibody fragments thereof. In some embodiments, the anti-human LAIR1 antibodies or antibody fragments thereof provided herein are agonists of LAIR1. In some embodiments, the anti-human LAIR1 antibodies provided herein are human antibodies, e.g., a human IgG2 or IgG4 isotype. In some embodiments, the anti-human LAIR1 antibodies provided herein also bind cynomolgus monkey LAIR1.

In some embodiments, provided herein are antibodies that bind human LAIR1, wherein the antibodies comprise a VH and a VL, wherein the VH comprises HCDR1, HCDR2, and HCDR3, and the VL comprises LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, the anti-human LAIR1 antibodies comprise a VH comprising SEQ ID NO: 7 and a VL comprising SEQ ID NO: 8. In some embodiments, the anti-human LAIR1 antibodies comprise a VH comprising a sequence having at least 95% sequence identity to SEQ ID NO: 7, and a VL comprising a sequence having at least 95% sequence identity to SEQ ID NO: 8.

In some embodiments, provided herein are antibodies that bind human LAIR1, wherein the antibodies comprise a VH and a VL, wherein the VH comprises HCDR1, HCDR2, and HCDR3, and the VL comprises LCDR1, LCDR2, and LCDR3, wherein HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 5, and LCDR3 comprises SEQ ID NO: 18. In some embodiments, the anti-human LAIR1 antibodies comprise a VH comprising SEQ ID NO: 19 and a VL comprising SEQ ID NO: 20. In some embodiments, the anti-human LAIR1 antibodies comprise a VH comprising a sequence having at least 95% sequence identity to SEQ ID NO: 19, and a VL comprising a sequence having at least 95% sequence identity to SEQ ID NO: 20.

In some embodiments, the anti-human LAIR1 antibody has a human IgG2 isotype. In some embodiments, the antibody comprises a heavy chain (HC) comprising SEQ ID NO: 9 and a light chain (LC) comprising SEQ ID NO: 10. In some embodiments, the antibody comprises a heavy chain comprising SEQ ID NO: 21 and a light chain comprising SEQ ID NO: 22.

In some embodiments, the anti-human LAIR1 antibody has a human IgG4 isotype. In some embodiments, the antibody comprises a heavy chain (HC) comprising SEQ ID NO: 25 and a light chain (LC) comprising SEQ ID NO: 10. In some embodiments, the antibody comprises a heavy chain comprising SEQ ID NO: 27 and a light chain comprising SEQ ID NO: 22.

In another aspect, provided herein are nucleic acids encoding a heavy chain or light chain, or a VH or VL, of the novel anti-human LAIR1 antibodies described herein, and vectors or cells comprising such nucleic acids.

In another aspect, provided herein are pharmaceutical compositions comprising an antibody, nucleic acid, or vector described herein.

The anti-human LAIR1 antibodies, nucleic acids, vectors, or pharmaceutical compositions described herein can be used for treating an autoimmune disease or fibrotic disease, e.g., rheumatoid arthritis, psoriasis, systemic lupus erythematosus (SLE), lupus nephritis, pemphigus vulgaris, systemic sclerosis, idiopathic pulmonary fibrosis, scleroderma, ulcerative colitis, Crohn's disease, hidradenitis suppurativa, atopic dermatitis, multiple sclerosis, scleroderma-associated interstitial lung disease, IgG4 related disease or chronic fibrosing interstitial lung diseases.

In another aspect, the antibody of the present invention is an antibody which does not form a complex with LAIR1 ligand C1q.

According to yet another aspect of the present invention, the antibody of the present invention increases the population of Treg cells in the spleen.

In yet further aspect of the present invention, the antibody of the present invention does not require full receptor occupancy (RO) to elicit agonism to human LAIR1.

### BRIEF DESCRIPTION OF THE DRAWINGS

FIGS. 1A-1F show the exemplified anti-human LAIR1 antibodies mAb1-mAb4 significantly inhibited the increase in plasma human pro-inflammatory cytokines IFN-γ (1A and 1D), TNF-α (1B and 1E) and IL-10 (1C and 1F) in the GvHD model at Day 7 (1A-1C) and Day 14 (1D-1F).

FIGS. 2A-2C show the exemplified anti-human LAIR1 antibodies mAb1-mAb3 significantly reduce circulating immunoglobulins IgM and IgA.

FIG. 3 shows that mAb4 inhibits TCR-stimulated NFAT activation in Jurkat-hLAIR1+ cells in vitro. A. Inhibition of NFAT activity following TCR stimulation reported as antibody-mediated inhibition % vs. NFAT activity in absence of antibody. B. mAb4 NFAT IC50 curve calculated and averaged from n=2 independent experiments.

FIG. 4 shows that mAb4 agonist antibody inhibits BCR-stimulated IL-6 response in human B cells in vitro. A. Inhibition of IL-6 response following BCR stimulation reported as antibody-mediated inhibition % vs. IL-6 response in absence of antibody. B. mAb4 B cell IL-6 IC50 curve calculated and averaged from n=3 independent experiments.

FIG. 5 shows mAb4 dose dependently and significantly inhibited serum human cytokine production compared to isotype control measured 8 days post engraftment (7 days post treatment). One-way ANOVA followed by Dunnett's post-test vs. Isotype (mean±SEM, n=7-8).

FIG. 6 shows Dose dependent receptor occupancy of mAb4 on 3 T cell subsets 7 days following a single SC dose.

FIG. 7 shows Dose dependent serum drug concentrations of mAb4 4 days (blue bars) and 7 days (red bars) following a single SC dose.

FIG. 8 shows mAb4 induced Treg expansion in a dose dependent manner.

FIG. 9 shows urine albumin-to-creatinine ratios (ACRs) of IFNα-induced NZB/W F1 mice treated with IgG isotype, surrogate antibody, or cyclophosphamide (CP) starting on Day 7 (D7) or Day 21 (D21). One-way ANOVA followed by Dunnett's post-test vs. IFNα-induced treated with IgG isotype, mean±SEM, n=5-10.

FIG. 10: shows Day 44 urine albumin-to-creatinine ratios (ACRs) of IFNα-induced NZB/W F1 mice treated with IgG isotype, surrogate antibody, or cyclophosphamide (CP) starting on Day 7 (D7) or Day 21 (D21). One-way ANOVA followed by Dunnett's post-test vs. IFNα-induced treated with IgG isotype, mean±SEM, n=5-10.

FIG. 11: shows kidney total histology scores of IFNα-induced NZB/W F1 mice treated with IgG isotype, surrogate antibody, or cyclophosphamide (CP) starting on Day 7 (D7) or Day 21 (D21). One-way ANOVA followed by Dunnett's post-test vs. IFNα-induced treated with IgG isotype, mean±SEM, n=5-10.

FIG. 12: shows that Ab4 and the humanized IgG4-P isotype control antibody had no binding to the complement component C1q. The anti-LAIR1 IgG1 antibody, and human IgG1 isotype control antibody, did bind complement component C1q as expected.

FIG. 13: shows selectivity for LAIR1 and no activity against LAIR2 by mAb4.

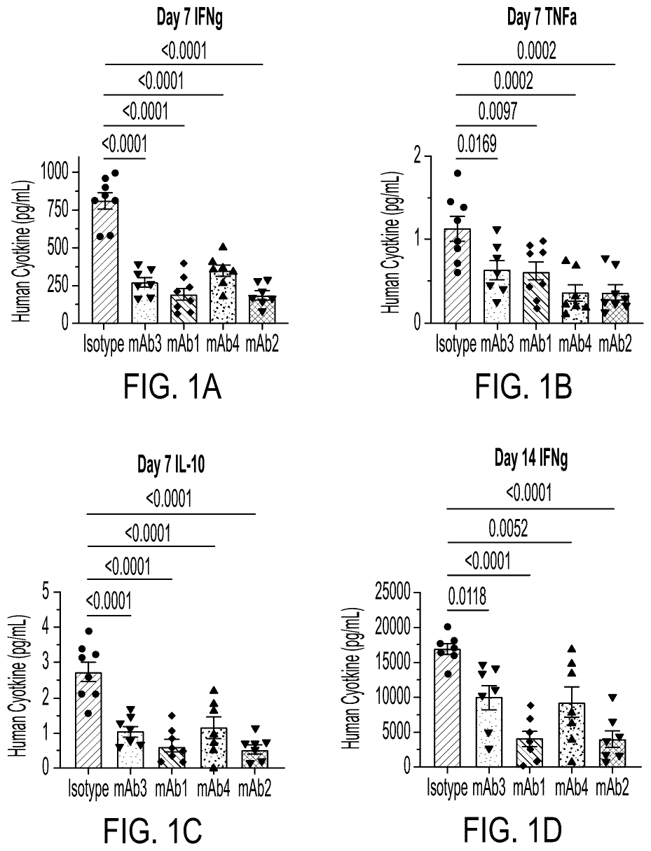

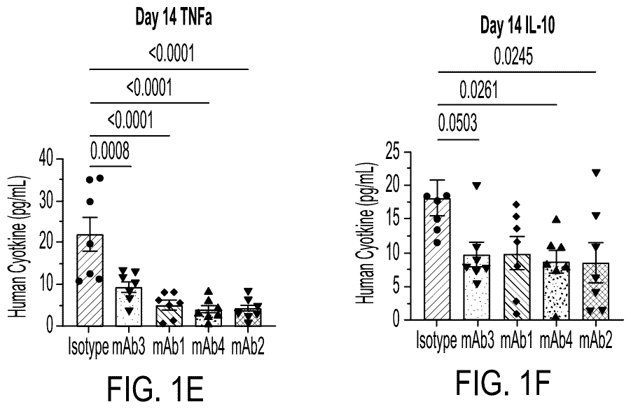

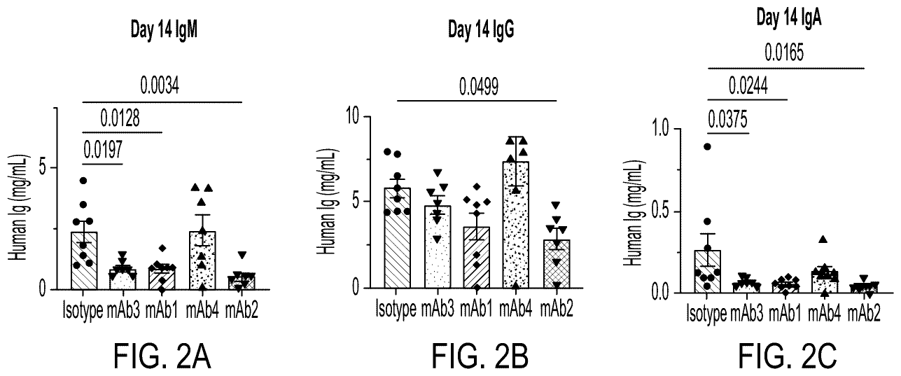

### DETAILED DESCRIPTION

Provided herein are antibodies that bind human LAIR1 (“anti-human LAIR1 antibodies” or “anti-human LAIR1 antibodies”), compositions comprising such anti-human LAIR1 antibodies, and methods of using such anti-human LAIR1 antibodies.

In one aspect, provided herein are novel anti-human LAIR1 antibodies or antibody fragments thereof. In some embodiments, the anti-human LAIR1 antibodies or antibody fragments thereof provided herein are agonists of LAIR1. In some embodiments, the anti-human LAIR1 antibodies or antibody fragments thereof provided herein can induce or increase one or more activities or functions associated with human LAIR1, e.g., one or more activities or functions described in the Examples. Such activities or functions associated with human LAIR1 include, but not limited to, inhibition of NFAT activation as determined by a Jurkat-NFAT activation assay, inhibition of IFN-g response in primary T cells following TCR stimulation assay, inhibition of IL-6 response in primary B cells following BCR stimulation assay, inhibition of increase in plasma human pro-inflammatory cytokines IFN-γ, IL-10, and TNF-α, and/or reduce circulating IgM and IgA in a human PBMC engrafted GvHD mouse model, as described in the Examples. In some embodiments, the anti-human LAIR1 antibodies provided herein do not block interaction between LAIR1 and its ligand, e.g., Collagen I.

In some embodiments, the anti-human LAIR1 antibodies provided herein are human antibodies, e.g., a human IgG2 or IgG4 isotype. In some embodiments, the anti-human LAIR1 antibodies provided herein also bind cynomolgus monkey LAIR1. In some embodiments, the anti-human LAIR1 antibodies provided herein have low immunogenicity risk.

In some embodiments, the anti-human LAIR1 antibody comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, the anti-human LAIR1 antibody comprises a VH comprising HCDR1, HCDR2, and HCDR3 selected from Table 1. In some embodiments, the anti-human LAIR1 antibody comprises a VL comprising LCDR1, LCDR2, and LCDR3 selected from Table 1. In some embodiments, the anti-human LAIR1 antibody comprises a VH comprising HCDR1, HCDR2, and HCDR3 selected from Table 1, and a VL comprising LCDR1, LCDR2, and LCDR3 selected from Table 1. In some embodiments, the anti-human LAIR1 antibody comprises a VH comprising a sequence having at least 95% sequence identity to a VH in Table 1. In some embodiments, the anti-human LAIR1 antibody comprises a VL comprising a sequence having at least 95% sequence identity to a VL in Table 1. In some embodiments, the anti-human LAIR1 antibody comprises a VH and/or a VL in Table 1.

TABLE 1
Sequences of exemplary anti-LAIR1 monoclonal antibodies

| SEQ | Ab | |
|---|---|---|
| ID NO | Region | Sequence |
| mAb1 | | |
| 1 | HCDR1 | TAFGGSISGHYWS |
| 2 | HCDR2 | RIYPSGGTN |
| 3 | HCDR3 | VRDYWDDYYGMDV |
| 4 | LCDR1 | RSSQSLLHSDGFNYLD |
| 5 | LCDR2 | YLGSNRAS |
| 6 | LCDR3 | MQAYQTPRT |
| 7 | VH | QVQLQESGPGLVKPSETLSLTCTAFGGSISGHYWSWIRQPAGKGLE |
| | | WIGRIYPSGGTNKNPSLKSRVTMSVDTSKNQFSLKLSSVTAADTAV |
| | | YYCVRDYWDDYYGMDVWGQGTTVTVSS |
| 8 | VL | DIVMTQSPLSLPVTPGEPASISCRSSQSLLHSDGFNYLDWYLQKPG |
| | | QSPQLLIYLGSNRASGVPDRFSGSGSGTDFTLKISRVEAEDVGVYY |
| | | CMQAYQTPRTFGQGTKVEIK |
| 9 | HC | QVQLQESGPGLVKPSETLSLTCTAFGGSISGHYWSWIRQPAGKGLE |
| | | WIGRIYPSGGTNKNPSLKSRVTMSVDTSKNQFSLKLSSVTAADTAV |
| | | YYCVRDYWDDYYGMDVWGQGTTVTVSSASTKGPSVFPLAPSSRS |
| | | TSESTAALGCLVKDYFPEPVTVSWNSGALTSGVHTFPAVLQSSGLY |
| | | SLSSVVTVPSSNFGTQTYTCNVDHKPSNTKVDKTVERKCCVECPPC |
| | | PAPPVAGPSVFLFPPKPKDTLMISRTPEVTCVVVDVSHEDPEVQFN |
| | | WYVDGMEVHNAKTKPREEQFNSTFRVVSVLTVVHQDWLNGKEY |
| | | KCKVSNKGLPAPIEKTISKTKGQPREPQVYTLPPSREEMTKNQVSL |
| | | TCLVKGFYPSDIAVEWESNGQPENNYKTTPPMLDSDGSFFLYSKLT |
| | | VDKSRWQQGNVFSCSVMHEALHNHYTQKSLSLSPG |
| 10 | LC | DIVMTQSPLSLPVTPGEPASISCRSSQSLLHSDGFNYLDWYLQKPG |
| | | QSPQLLIYLGSNRASGVPDRFSGSGSGTDFTLKISRVEAEDVGVYY |
| | | CMQAYQTPRTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 11 | HC DNA | CAGGTGCAGCTGCAGGAGTCGGGCCCAGGACTGGTGAAGCCTT |
| | | CGGAGACCCTGTCCCTCACCTGTACTGCCTTTGGTGGCTCCATC |
| | | AGTGGTCACTACTGGAGCTGGATCCGGCAGCCCGCCGGGAAGG |
| | | GACTGGAGTGGATTGGGCGTATTTATCCTAGTGGGGGCACCAA |
| | | CAAAAACCCCTCCCTCAAGAGTCGCGTCACCATGTCAGTAGAC |
| | | ACGTCCAAGAATCAGTTCTCCCTGAAGCTGAGTTCTGTGACCGC |
| | | CGCGGACACGGCCGTGTATTATTGTGTGAGAGATTACTGGGAC |
| | | GACTACTATGGTATGGACGTCTGGGGCCAAGGGACCACGGTCA |
| | | CCGTCTCCTCAGCCTCCACCAAGGGCCCATCGGTCTTCCCCCTG |
| | | GCGCCCAGCTCCAGGAGCACCTCCGAGAGCACAGCCGCCCTGG |
| | | GCTGCCTGGTCAAGGACTACTTCCCCGAACCGGTGACGGTGTCG |
| | | TGGAACTCAGGCGCCCTGACCAGCGGCGTGCACACCTTCCCGG |
| | | CTGTCCTACAGTCCTCAGGACTCTACTCCCTCAGCAGCGTGGTG |
| | | ACCGTGCCCTCCAGCAACTTCGGCACCCAGACCTACACCTGCAA |
| | | CGTAGATCACAAGCCCAGCAACACCAAGGTGGACAAGACAGTT |
| | | GAGCGCAAATGTTGTGTCGAGTGCCCACCGTGCCCAGCACCAC |
| | | CTGTGGCAGGACCGTCAGTCTTCCTCTTCCCCCCAAAACCCAAG |
| | | GACACCCTCATGATCTCCCGGACCCCTGAGGTCACGTGCGTGGT |
| | | GGTGGACGTGAGCCACGAAGACCCCGAGGTCCAGTTCAACTGG |
| | | TACGTGGACGGCATGGAGGTGCATAATGCCAAGACAAAGCCAC |
| | | GGGAGGAGCAGTTCAACAGCACGTTCCGTGTGGTCAGCGTCCT |
| | | CACCGTCGTGCACCAGGACTGGCTGAACGGCAAGGAGTACAAG |
| | | TGCAAGGTCTCCAACAAAGGCCTCCCAGCCCCCATCGAGAAAA |
| | | CCATCTCCAAAACCAAAGGGCAGCCCCGAGAACCACAGGTGTA |
| | | CACCCTGCCCCCATCCCGGGAGGAGATGACCAAGAACCAGGTC |
| | | AGCCTGACCTGCCTGGTCAAAGGCTTCTACCCCAGCGACATCGC |
| | | CGTGGAGTGGGAGAGCAATGGGCAGCCGGAGAACAACTACAA |
| | | GACCACACCTCCCATGCTGGACTCCGACGGCTCCTTCTTCCTCT |
| | | ACAGCAAGCTCACCGTGGACAAGAGCAGGTGGCAGCAGGGGA |
| | | ACGTCTTCTCATGCTCCGTGATGCATGAGGCTCTGCACAACCAC |
| | | TACACACAGAAGAGCCTCTCCCTGTCTCCGGGTTGA |
| 12 | LC DNA | GATATTGTGATGACTCAGTCTCCACTCTCCCTGCCCGTCACCCCT |
| | | GGAGAGCCGGCCTCCATCTCCTGCAGGTCTAGTCAGAGCCTCCT |
| | | GCATAGTGATGGATTCAACTATTTGGATTGGTACCTGCAGAAGC |
| | | CAGGGCAGTCTCCACAGCTCCTGATCTATCTGGGTTCTAATCGG |
| | | GCCTCCGGGGTCCCTGACAGGTTCAGTGGCAGTGGATCAGGCA |
| | | CAGATTTTACACTGAAAATCAGCAGAGTGGAGGCTGAGGATGT |
| | | TGGGGTTTATTACTGCATGCAAGCTTATCAAACTCCTCGGACGT |
| | | TCGGCCAAGGGACCAAGGTGGAAATCAAACGAACCGTGGCTGC |
| | | ACCATCTGTCTTCATCTTCCCGCCATCTGATGAGCAGTTGAAAT |
| | | CTGGAACTGCCTCTGTTGTGTGCCTGCTGAATAACTTCTATCCC |
| | | AGAGAGGCCAAAGTACAGTGGAAGGTGGATAACGCCCTCCAAT |
| | | CGGGTAACTCCCAGGAGAGTGTCACAGAGCAGGACAGCAAGGA |
| | | CAGCACCTACAGCCTCAGCAGCACCCTGACGCTGAGCAAAGCA |
| | | GACTACGAGAAACACAAAGTCTACGCCTGCGAAGTCACCCATC |
| | | AGGGCCTGAGCTCGCCCGTCACAAAGAGCTTCAACAGGGGAGA |
| | | GTGC |
| mAb2 | | |
| 13 | HCDR1 | TVSGGSISNNYWS |
| 14 | HCDR2 | RMYSSGSTQ |
| 15 | HCDR3 | GPNWGSPYYGVDV |
| 16 | LCDR1 | RSSPSLLHSDGYNWLD |
| 5 | LCDR2 | YLGSNRAS |
| 18 | LCDR3 | MQAHQTPFT |
| 19 | VH | QVQLQESGPGLVKPSETLSLTCTVSGGSISNNYWSWIRQPAGKGLE |
| | | WIGRMYSSGSTQKNPSLKSRVTMSVDTSKNQFSLKLSSVTAADTA |
| | | VYYCVRGPNWGSPYYGVDVWGQGTTVTVSS |
| 20 | VL | DIVMTQSPLSLPVTPGEPASISCRSSPSLLHSDGYNWLDWYLQKPG |
| | | QSPQLLIYLGSNRASGVPDRFSGSGSGTDFTLKISRVEAEDVGVYY |
| | | CMQAHQTPFTFGQGTKVEIK |
| 21 | HC | QVQLQESGPGLVKPSETLSLTCTVSGGSISNNYWSWIRQPAGKGLE |
| | | WIGRMYSSGSTQKNPSLKSRVTMSVDTSKNQFSLKLSSVTAADTA |
| | | VYYCVRGPNWGSPYYGVDVWGQGTTVTVSSASTKGPSVFPLAPS |
| | | SRSTSESTAALGCLVKDYFPEPVTVSWNSGALTSGVHTFPAVLQSS |
| | | GLYSLSSVVTVPSSNFGTQTYTCNVDHKPSNTKVDKTVERKCCVE |
| | | CPPCPAPPVAGPSVFLFPPKPKDTLMISRTPEVTCVVVDVSHEDPEV |
| | | QFNWYVDGMEVHNAKTKPREEQFNSTFRVVSVLTVVHQDWLNG |
| | | KEYKCKVSNKGLPAPIEKTISKTKGQPREPQVYTLPPSREEMTKNQ |
| | | VSLTCLVKGFYPSDIAVEWESNGQPENNYKTTPPMLDSDGSFFLYS |
| | | KLTVDKSRWQQGNVFSCSVMHEALHNHYTQKSLSLSPG |
| 22 | LC | DIVMTQSPLSLPVTPGEPASISCRSSPSLLHSDGYNWLDWYLQKPG |
| | | QSPQLLIYLGSNRASGVPDRFSGSGSGTDFTLKISRVEAEDVGVYY |
| | | CMQAHQTPFTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 23 | HC DNA | CAGGTGCAGCTGCAGGAGTCGGGCCCAGGACTGGTGAAGCCTT |
| | | CGGAGACCCTGTCCCTCACCTGCACTGTCTCTGGTGGCTCCATC |
| | | AGTAATAACTACTGGAGTTGGATCCGGCAGCCCGCCGGTAAGG |
| | | GGCTGGAGTGGATTGGGCGTATGTATTCCAGTGGGAGCACCCA |
| | | GAAAAACCCCTCCCTCAAGAGTCGAGTCACCATGTCAGTAGAC |
| | | ACGTCCAAAAACCAGTTCTCCCTGAAGCTGTCCTCTGTGACCGC |
| | | CGCGGACACGGCCGTGTATTATTGTGTGAGAGGCCCTAACTGG |
| | | GGATCCCCCTACTACGGTGTGGACGTCTGGGGCCAAGGGACCA |
| | | CGGTCACCGTCTCCTCAGCCTCCACCAAGGGCCCATCGGTCTTC |
| | | CCCCTGGCGCCCAGCTCCAGGAGCACCTCCGAGAGCACAGCCG |
| | | CCCTGGGCTGCCTGGTCAAGGACTACTTCCCCGAACCGGTGACG |
| | | GTGTCGTGGAACTCAGGCGCCCTGACCAGCGGCGTGCACACCTT |
| | | CCCGGCTGTCCTACAGTCCTCAGGACTCTACTCCCTCAGCAGCG |
| | | TGGTGACCGTGCCCTCCAGCAACTTCGGCACCCAGACCTACACC |
| | | TGCAACGTAGATCACAAGCCCAGCAACACCAAGGTGGACAAGA |
| | | CAGTTGAGCGCAAATGTTGTGTCGAGTGCCCACCGTGCCCAGCA |
| | | CCACCTGTGGCAGGACCGTCAGTCTTCCTCTTCCCCCCAAAACC |
| | | CAAGGACACCCTCATGATCTCCCGGACCCCTGAGGTCACGTGCG |
| | | TGGTGGTGGACGTGAGCCACGAAGACCCCGAGGTCCAGTTCAA |
| | | CTGGTACGTGGACGGCATGGAGGTGCATAATGCCAAGACAAAG |
| | | CCACGGGAGGAGCAGTTCAACAGCACGTTCCGTGTGGTCAGCG |
| | | TCCTCACCGTCGTGCACCAGGACTGGCTGAACGGCAAGGAGTA |
| | | CAAGTGCAAGGTCTCCAACAAAGGCCTCCCAGCCCCCATCGAG |
| | | AAAACCATCTCCAAAACCAAAGGGCAGCCCCGAGAACCACAGG |
| | | TGTACACCCTGCCCCCATCCCGGGAGGAGATGACCAAGAACCA |
| | | GGTCAGCCTGACCTGCCTGGTCAAAGGCTTCTACCCCAGCGACA |
| | | TCGCCGTGGAGTGGGAGAGCAATGGGCAGCCGGAGAACAACTA |
| | | CAAGACCACACCTCCCATGCTGGACTCCGACGGCTCCTTCTTCC |
| | | TCTACAGCAAGCTCACCGTGGACAAGAGCAGGTGGCAGCAGGG |
| | | GAACGTCTTCTCATGCTCCGTGATGCATGAGGCTCTGCACAACC |
| | | ACTACACACAGAAGAGCCTCTCCCTGTCTCCGGGTTGA |
| 24 | LC DNA | GATATTGTGATGACTCAGTCTCCACTCTCCCTGCCCGTCACCCCT |
| | | GGAGAGCCGGCCTCCATCTCCTGCAGGTCTAGTCCGAGCCTCCT |
| | | GCATAGTGATGGATACAACTGGTTGGATTGGTACCTGCAGAAG |
| | | CCAGGGCAGTCTCCACAGCTCCTGATCTATTTGGGTTCTAATCG |
| | | GGCCTCCGGGGTCCCTGACAGGTTCAGTGGCAGTGGATCAGGC |
| | | ACAGATTTTACACTGAAAATCAGCAGAGTGGAGGCTGAGGATG |
| | | TTGGGGTTTATTACTGCATGCAAGCTCACCAAACTCCATTCACT |
| | | TTCGGCCAGGGGACCAAGGTGGAAATCAAACGAACCGTGGCTG |
| | | CACCATCTGTCTTCATCTTCCCGCCATCTGATGAGCAGTTGAAA |
| | | TCTGGAACTGCCTCTGTTGTGTGCCTGCTGAATAACTTCTATCCC |
| | | AGAGAGGCCAAAGTACAGTGGAAGGTGGATAACGCCCTCCAAT |
| | | CGGGTAACTCCCAGGAGAGTGTCACAGAGCAGGACAGCAAGGA |
| | | CAGCACCTACAGCCTCAGCAGCACCCTGACGCTGAGCAAAGCA |
| | | GACTACGAGAAACACAAAGTCTACGCCTGCGAAGTCACCCATC |
| | | AGGGCCTGAGCTCGCCCGTCACAAAGAGCTTCAACAGGGGAGA |
| | | GTGC |
| mAb3 | | |
| 1 | HCDR1 | TAFGGSISGHYWS |
| 2 | HCDR2 | RIYPSGGTN |
| 3 | HCDR3 | VRDYWDDYYGMDV |
| 4 | LCDR1 | RSSQSLLHSDGFNYLD |
| 5 | LCDR2 | YLGSNRAS |
| 6 | LCDR3 | MQAYQTPRT |
| 7 | VH | QVQLQESGPGLVKPSETLSLTCTAFGGSISGHYWSWIRQPAGKGLE |
| | | WIGRIYPSGGTNKNPSLKSRVTMSVDTSKNQFSLKLSSVTAADTAV |
| | | YYCVRDYWDDYYGMDVWGQGTTVTVSS |
| 8 | VL | DIVMTQSPLSLPVTPGEPASISCRSSQSLLHSDGFNYLDWYLQKPG |
| | | QSPQLLIYLGSNRASGVPDRFSGSGSGTDFTLKISRVEAEDVGVYY |
| | | CMQAYQTPRTFGQGTKVEIK |
| 25 | HC | QVQLQESGPGLVKPSETLSLTCTAFGGSISGHYWSWIRQPAGKGLE |
| | | WIGRIYPSGGTNKNPSLKSRVTMSVDTSKNQFSLKLSSVTAADTAV |
| | | YYCVRDYWDDYYGMDVWGQGTTVTVSSASTKGPSVFPLAPCSRS |
| | | TSESTAALGCLVKDYFPEPVTVSWNSGALTSGVHTFPAVLQSSGLY |
| | | SLSSVVTVPSSSLGTKTYTCNVDHKPSNTKVDKRVESKYGPPCPPC |
| | | PAPEFLGGPSVFLFPPKPKDTLMISRTPEVTCVVVDVSQEDPEVQFN |
| | | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLT |
| | | VDKSRWQEGNVFSCSVMHEALHNHYTQKSLSLSLG |
| 10 | LC | DIVMTQSPLSLPVTPGEPASISCRSSQSLLHSDGFNYLDWYLQKPG |
| | | QSPQLLIYLGSNRASGVPDRFSGSGSGTDFTLKISRVEAEDVGVYY |
| | | CMQAYQTPRTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 26 | HC DNA | CAGGTGCAGCTGCAGGAGTCGGGCCCAGGACTGGTGAAGCCTT |
| | | CGGAGACCCTGTCCCTCACCTGTACTGCCTTTGGTGGCTCCATC |
| | | AGTGGTCACTACTGGAGCTGGATCCGGCAGCCCGCCGGGAAGG |
| | | GACTGGAGTGGATTGGGCGTATTTATCCTAGTGGGGGCACCAA |
| | | CAAAAACCCCTCCCTCAAGAGTCGCGTCACCATGTCAGTAGAC |
| | | ACGTCCAAGAATCAGTTCTCCCTGAAGCTGAGTTCTGTGACCGC |
| | | CGCGGACACGGCCGTGTATTATTGTGTGAGAGATTACTGGGAC |
| | | GACTACTATGGTATGGACGTCTGGGGCCAAGGGACCACGGTCA |
| | | CCGTCTCCTCAGCTAGCACCAAGGGCCCATCGGTCTTCCCACTA |
| | | GCGCCCTGCTCCAGGAGCACCTCCGAGAGCACAGCCGCCCTGG |
| | | GCTGCCTGGTCAAGGACTACTTCCCCGAACCGGTGACGGTGTCG |
| | | TGGAACTCAGGCGCCCTGACCAGCGGCGTGCACACCTTCCCGG |
| | | CTGTCCTACAGTCCTCAGGACTCTACTCCCTCAGCAGCGTGGTG |
| | | ACCGTGCCCTCCAGCAGCTTGGGCACGAAGACCTACACCTGCA |
| | | ACGTAGATCACAAGCCCAGCAACACCAAGGTGGACAAGAGAGT |
| | | TGAGTCCAAATATGGTCCCCCATGCCCACCCTGCCCAGCACCTG |
| | | AGTTTCTGGGGGGACCATCAGTCTTCCTGTTCCCCCCAAAACCC |
| | | AAGGACACTCTCATGATCTCCCGGACCCCTGAGGTCACGTGCGT |
| | | GGTGGTGGACGTGAGCCAGGAAGACCCCGAGGTCCAGTTCAAC |
| | | TGGTACGTGGATGGCGTGGAGGTGCATAATGCCAAGACAAAGC |
| | | CGCGGGAGGAGCAGTTCAACAGCACGTACCGTGTGGTCAGCGT |
| | | CCTCACCGTCCTGCACCAGGACTGGCTGAACGGCAAGGAGTAC |
| | | AAGTGCAAGGTCTCCAACAAAGGCCTCCCGTCCTCCATCGAGA |
| | | AAACCATCTCCAAAGCCAAAGGGCAGCCCCGAGAGCCACAGGT |
| | | GTACACCCTGCCCCCATCCCAGGAGGAGATGACCAAGAACCAG |
| | | GTCAGCCTGACCTGCCTGGTCAAAGGCTTCTACCCCAGCGACAT |
| | | CGCCGTGGAGTGGGAAAGCAATGGGCAGCCGGAGAACAACTAC |
| | | AAGACCACGCCTCCCGTGCTGGACTCCGACGGCTCCTTCTTCCT |
| | | CTACAGCAGGCTAACCGTGGACAAGAGCAGGTGGCAGGAGGG |
| | | GAATGTCTTCTCATGCTCCGTGATGCATGAGGCTCTGCACAACC |
| | | ACTACACACAGAAGAGCCTCTCCCTGTCTCTGGGT |
| 12 | LC DNA | GATATTGTGATGACTCAGTCTCCACTCTCCCTGCCCGTCACCCCT |
| | | GGAGAGCCGGCCTCCATCTCCTGCAGGTCTAGTCAGAGCCTCCT |
| | | GCATAGTGATGGATTCAACTATTTGGATTGGTACCTGCAGAAGC |
| | | CAGGGCAGTCTCCACAGCTCCTGATCTATCTGGGTTCTAATCGG |
| | | GCCTCCGGGGTCCCTGACAGGTTCAGTGGCAGTGGATCAGGCA |
| | | CAGATTTTACACTGAAAATCAGCAGAGTGGAGGCTGAGGATGT |
| | | TGGGGTTTATTACTGCATGCAAGCTTATCAAACTCCTCGGACGT |
| | | TCGGCCAAGGGACCAAGGTGGAAATCAAACGAACCGTGGCTGC |
| | | ACCATCTGTCTTCATCTTCCCGCCATCTGATGAGCAGTTGAAAT |
| | | CTGGAACTGCCTCTGTTGTGTGCCTGCTGAATAACTTCTATCCC |
| | | AGAGAGGCCAAAGTACAGTGGAAGGTGGATAACGCCCTCCAAT |
| | | CGGGTAACTCCCAGGAGAGTGTCACAGAGCAGGACAGCAAGGA |
| | | CAGCACCTACAGCCTCAGCAGCACCCTGACGCTGAGCAAAGCA |
| | | GACTACGAGAAACACAAAGTCTACGCCTGCGAAGTCACCCATC |
| | | AGGGCCTGAGCTCGCCCGTCACAAAGAGCTTCAACAGGGGAGA |
| | | GTGC |
| mAb4 | | |
| 13 | HCDR1 | TVSGGSISNNYWS |
| 14 | HCDR2 | RMYSSGSTQ |
| 15 | HCDR3 | VRGPNWGSPYYGVDV |
| 16 | LCDR1 | RSSPSLLHSDGYNWLD |
| 5 | LCDR2 | YLGSNRAS |
| 18 | LCDR3 | MQAHQTPFT |
| 19 | VH | QVQLQESGPGLVKPSETLSLTCTVSGGSISNNYWSWIRQPAGKGLE |
| | | WIGRMYSSGSTQKNPSLKSRVTMSVDTSKNQFSLKLSSVTAADTA |
| | | VYYCVRGPNWGSPYYGVDVWGQGTTVTVSS |
| 20 | VL | DIVMTQSPLSLPVTPGEPASISCRSSPSLLHSDGYNWLDWYLQKPG |
| | | QSPQLLIYLGSNRASGVPDRFSGSGSGTDFTLKISRVEAEDVGVYY |
| | | CMQAHQTPFTFGQGTKVEIK |
| 27 | HC | QVQLQESGPGLVKPSETLSLTCTVSGGSISNNYWSWIRQPAGKGLE |
| | | WIGRMYSSGSTQKNPSLKSRVTMSVDTSKNQFSLKLSSVTAADTA |
| | | VYYCVRGPNWGSPYYGVDVWGQGTTVTVSSASTKGPSVFPLAPC |
| | | SRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYS |
| | | RLTVDKSRWQEGNVFSCSVMHEALHNHYTQKSLSLSLG |
| 22 | LC | DIVMTQSPLSLPVTPGEPASISCRSSPSLLHSDGYNWLDWYLQKPG |
| | | QSPQLLIYLGSNRASGVPDRFSGSGSGTDFTLKISRVEAEDVGVYY |
| | | CMQAHQTPFTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 28 | HC DNA | CAGGTGCAGCTGCAGGAGTCGGGCCCAGGACTGGTGAAGCCTT |
| | | CGGAGACCCTGTCCCTCACCTGCACTGTCTCTGGTGGCTCCATC |
| | | AGTAATAACTACTGGAGTTGGATCCGGCAGCCCGCCGGTAAGG |
| | | GGCTGGAGTGGATTGGGCGTATGTATTCCAGTGGGAGCACCCA |
| | | GAAAAACCCCTCCCTCAAGAGTCGAGTCACCATGTCAGTAGAC |
| | | ACGTCCAAAAACCAGTTCTCCCTGAAGCTGTCCTCTGTGACCGC |
| | | CGCGGACACGGCCGTGTATTATTGTGTGAGAGGCCCTAACTGG |
| | | GGATCCCCCTACTACGGTGTGGACGTCTGGGGCCAAGGGACCA |
| | | CGGTCACCGTCTCCTCAGCTAGCACCAAGGGCCCATCGGTCTTC |
| | | CCACTAGCGCCCTGCTCCAGGAGCACCTCCGAGAGCACAGCCG |
| | | CCCTGGGCTGCCTGGTCAAGGACTACTTCCCCGAACCGGTGACG |
| | | GTGTCGTGGAACTCAGGCGCCCTGACCAGCGGCGTGCACACCTT |
| | | CCCGGCTGTCCTACAGTCCTCAGGACTCTACTCCCTCAGCAGCG |
| | | TGGTGACCGTGCCCTCCAGCAGCTTGGGCACGAAGACCTACAC |
| | | CTGCAACGTAGATCACAAGCCCAGCAACACCAAGGTGGACAAG |
| | | AGAGTTGAGTCCAAATATGGTCCCCCATGCCCACCCTGCCCAGC |
| | | ACCTGAGTTTCTGGGGGGACCATCAGTCTTCCTGTTCCCCCCAA |
| | | AACCCAAGGACACTCTCATGATCTCCCGGACCCCTGAGGTCACG |
| | | TGCGTGGTGGTGGACGTGAGCCAGGAAGACCCCGAGGTCCAGT |
| | | TCAACTGGTACGTGGATGGCGTGGAGGTGCATAATGCCAAGAC |
| | | AAAGCCGCGGGAGGAGCAGTTCAACAGCACGTACCGTGTGGTC |
| | | AGCGTCCTCACCGTCCTGCACCAGGACTGGCTGAACGGCAAGG |
| | | AGTACAAGTGCAAGGTCTCCAACAAAGGCCTCCCGTCCTCCATC |
| | | GAGAAAACCATCTCCAAAGCCAAAGGGCAGCCCCGAGAGCCAC |
| | | AGGTGTACACCCTGCCCCCATCCCAGGAGGAGATGACCAAGAA |
| | | CCAGGTCAGCCTGACCTGCCTGGTCAAAGGCTTCTACCCCAGCG |
| | | ACATCGCCGTGGAGTGGGAAAGCAATGGGCAGCCGGAGAACA |
| | | ACTACAAGACCACGCCTCCCGTGCTGGACTCCGACGGCTCCTTC |
| | | TTCCTCTACAGCAGGCTAACCGTGGACAAGAGCAGGTGGCAGG |
| | | AGGGGAATGTCTTCTCATGCTCCGTGATGCATGAGGCTCTGCAC |
| | | AACCACTACACACAGAAGAGCCTCTCCCTGTCTCTGGGT |
| 24 | LC DNA | GATATTGTGATGACTCAGTCTCCACTCTCCCTGCCCGTCACCCCT |
| | | GGAGAGCCGGCCTCCATCTCCTGCAGGTCTAGTCCGAGCCTCCT |
| | | GCATAGTGATGGATACAACTGGTTGGATTGGTACCTGCAGAAG |
| | | CCAGGGCAGTCTCCACAGCTCCTGATCTATTTGGGTTCTAATCG |
| | | GGCCTCCGGGGTCCCTGACAGGTTCAGTGGCAGTGGATCAGGC |
| | | ACAGATTTTACACTGAAAATCAGCAGAGTGGAGGCTGAGGATG |
| | | TTGGGGTTTATTACTGCATGCAAGCTCACCAAACTCCATTCACT |
| | | TTCGGCCAGGGGACCAAGGTGGAAATCAAACGAACCGTGGCTG |
| | | CACCATCTGTCTTCATCTTCCCGCCATCTGATGAGCAGTTGAAA |
| | | TCTGGAACTGCCTCTGTTGTGTGCCTGCTGAATAACTTCTATCCC |
| | | AGAGAGGCCAAAGTACAGTGGAAGGTGGATAACGCCCTCCAAT |
| | | CGGGTAACTCCCAGGAGAGTGTCACAGAGCAGGACAGCAAGGA |
| | | CAGCACCTACAGCCTCAGCAGCACCCTGACGCTGAGCAAAGCA |
| | | GACTACGAGAAACACAAAGTCTACGCCTGCGAAGTCACCCATC |
| | | AGGGCCTGAGCTCGCCCGTCACAAAGAGCTTCAACAGGGGAGA |
| | | GTGC |
| Ab0 | | |
| (Parental | | |
| Ab4) | | |
| 34 | HCDR1 | TVSGGSISNNYWS |
| 35 | HCDR2 | RMYSSGSTN |
| 36 | HCDR3 | ARGPNWGSPYYGVDV |
| 37 | LCDR1 | RSSQSLLHSDGYNYLD |
| 5 | LCDR2 | YLGSNRAS |
| 38 | LCDR3 | MQALQTPFT |
| 39 | HC | QVQLQESGPGLVKPSETLSLTCTVSGGSISNNYWSWIRQSAGKGLE |
| | | WIGRMYSSGSTNKNPSLKSRVTMSVDTSKNQFSLKLTSVTAADTA |
| | | VYYCARGPNWGSPYYGVDVWGQGTTVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 40 | LC | DVVMTQSPLSLPVTPGEPASISCRSSQSLLHSDGYNYLDWYLQKPG |
| | | QSPQVLIYLGSNRASGVPDRFSGSGSGTDFTLKISRVEAEDVGVYY |
| | | CMQALQTPFTFGPGTKVEIKRTVAAPSVFIFPPSDEQLKSGTASVVC |
| | | LLNNFYPREAKVQWKVDNALQSGNSQESVTEQDSKDSTYSLSSTL |
| | | TLSKADYEKHKVYACEVTHQGLSSPVTKSFNRGEC |

In some embodiments, provided herein are antibodies that bind human LAIR1, wherein the antibodies comprise a VH and a VL, wherein the VH comprises HCDR1, HCDR2, and HCDR3, and the VL comprises LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, the anti-human LAIR1 antibodies comprise a VH comprising SEQ ID NO: 7 and a VL comprising SEQ ID NO: 8. In some embodiments, the anti-human LAIR1 antibodies comprise a VH comprising a sequence having at least 95% sequence identity to SEQ ID NO: 7, and a VL comprising a sequence having at least 95% sequence identity to SEQ ID NO: 8.

In some embodiments, provided herein are antibodies that bind human LAIR1, wherein the antibodies comprise a VH and a VL, wherein the VH comprises HCDR1, HCDR2, and HCDR3, and the VL comprises LCDR1, LCDR2, and LCDR3, wherein HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 5, and LCDR3 comprises SEQ ID NO: 18. In some embodiments, the anti-human LAIR1 antibodies comprise a VH comprising SEQ ID NO: 19 and a VL comprising SEQ ID NO: 20. In some embodiments, the anti-human LAIR1 antibodies comprise a VH comprising a sequence having at least 95% sequence identity to SEQ ID NO: 19, and a VL comprising a sequence having at least 95% sequence identity to SEQ ID NO: 20.

In some embodiments, the anti-human LAIR1 antibody is a human antibody. In some embodiments, the anti-human LAIR1 antibody has a human IgG2 or IgG4 isotype. In some embodiments, the anti-human LAIR1 antibody has a human IgG2 isotype. In some embodiments, the anti-human LAIR1 antibody has a modified human IgG2 Fc region comprising a C131S mutation (according to the EU Index Numbering), which reduces disulfide bond heterogeneity in human IgG2 (see Allen, et al., Biochemistry 2009, 48: 3755-3766). In some embodiments, the anti-human LAIR1 antibody has a human IgG4 isotype. In some embodiments, the anti-human LAIR1 antibody has a modified human IgG4 hinge region comprising a S228P mutation (according to the EU Index Numbering), which reduces the IgG4 Fab-arm exchange in vivo (see Labrijn, et al., Nat. Biotechnol. 2009, 27(8):767).

In some embodiments, the anti-human LAIR1 antibody has a human IgG2 isotype. In some embodiments, the antibody comprises a heavy chain (HC) comprising SEQ ID NO: 9 and a light chain (LC) comprising SEQ ID NO: 10. In some embodiments, the antibody comprises a heavy chain comprising SEQ ID NO: 21 and a light chain comprising SEQ ID NO: 22.

In some embodiments, the anti-human LAIR1 antibody has a human IgG4 isotype. In some embodiments, the antibody comprises a heavy chain (HC) comprising SEQ ID NO: 25 and a light chain (LC) comprising SEQ ID NO: 10. In some embodiments, the antibody comprises a heavy chain comprising SEQ ID NO: 27 and a light chain comprising SEQ ID NO: 22.

In some embodiments, provided herein are antibody fragments (e.g., Fab or scFv) that bind human LAIR1, wherein the antibody fragments comprise a VH and a VL, wherein the VH comprises HCDR1, HCDR2, and HCDR3, and the VL comprises LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, the antibody fragments comprise a VH comprising SEQ ID NO: 7 and a VL comprising SEQ ID NO: 8.

In some embodiments, provided herein are antibody fragments (e.g., Fab or scFv) that bind human LAIR1, wherein the antibody fragments comprise a VH and a VL, wherein the VH comprises HCDR1, HCDR2, and HCDR3, and the VL comprises LCDR1, LCDR2, and LCDR3, wherein HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 5, and LCDR3 comprises SEQ ID NO: 18. In some embodiments, the antibody fragments comprise a VH comprising SEQ ID NO: 19 and a VL comprising SEQ ID NO: 20.

In another aspect, provided herein are nucleic acids encoding a heavy chain or light chain, or a VH or VL, of the novel anti-human LAIR1 antibodies described herein, and vectors comprising such nucleic acids.

In some embodiments, provided herein are nucleic acids encoding a heavy chain or light chain of the anti-human LAIR1 antibodies described herein. In some embodiments, provided herein are nucleic acids comprising a sequence encoding SEQ ID NO: 9, 25, 10, 21, 27 or 22. In some embodiments, provided herein are nucleic acids comprising a sequence encoding an antibody heavy chain that comprises SEQ ID NO: 9, 25, 21, or 27. For example, the nucleic acid can comprise a sequence selected from SEQ ID NO: 11, 26, 23, or 28. In some embodiments, provided herein are nucleic acids comprising a sequence encoding an antibody light chain that comprises SEQ ID NO: 10 or 22. For example, the nucleic acid can comprise a sequence selected from SEQ ID NO: 12 or 24.

Provided herein are also vectors comprising a nucleic acid sequence encoding an antibody heavy chain or light chain. For example, such vectors can comprise a nucleic acid sequence encoding SEQ ID NO: 9, 25, 10, 21, 27 or 22. In some embodiments, the vector comprises SEQ ID NO: 11, 26, 12, 23, 28 or 24.

Provided herein are also vectors comprising a first nucleic acid sequence encoding an antibody heavy chain and a second nucleic acid sequence encoding an antibody light chain. In some embodiments, the vector comprises a first nucleic acid sequence encoding SEQ ID NO: 9 or 25, and a second nucleic acid sequence encoding SEQ ID NO: 10. In some embodiments, the vector comprises a first nucleic acid sequence encoding SEQ ID NO: 21 or 27, and a second nucleic acid sequence encoding SEQ ID NO: 22.

Also provided are compositions comprising a first vector comprising a nucleic acid sequence encoding an antibody heavy chain, and a second vector comprising a nucleic acid sequence encoding an antibody light chain. In some embodiments, the composition comprises a first vector comprising a nucleic acid sequence encoding SEQ ID NO: 9 or 25, and a second vector comprising a nucleic acid sequence encoding SEQ ID NO: 10. In some embodiments, the composition comprises a first vector comprising a nucleic acid sequence encoding SEQ ID NO: 21 or 27, and a second vector comprising a nucleic acid sequence encoding SEQ ID NO: 22.

Nucleic acids of the present disclosure may be expressed in a host cell, for example, after the nucleic acids have been operably linked to an expression control sequence. Expression control sequences capable of expression of nucleic acids to which they are operably linked are well known in the art. An expression vector may include a sequence that encodes one or more signal peptides that facilitate secretion of the polypeptide(s) from a host cell. Expression vectors containing a nucleic acid of interest (e.g., a nucleic acid encoding a heavy chain or light chain of an antibody) may be transferred into a host cell by well-known methods, e.g., stable or transient transfection, transformation, transduction or infection. Additionally, expression vectors may contain one or more selection markers, e.g., tetracycline, neomycin, and dihydrofolate reductase, to aid in detection of host cells transformed with the desired nucleic acid sequences.

In another aspect, provided herein are cells, e.g., host cells, comprising the nucleic acids, vectors, or nucleic acid compositions described herein. A host cell may be a cell stably or transiently transfected, transformed, transduced or infected with one or more expression vectors expressing all or a portion of an antibody described herein. In some embodiments, a host cell may be stably or transiently transfected, transformed, transduced or infected with an expression vector expressing HC and LC polypeptides of an antibody of the present disclosure. In some embodiments, a host cell may be stably or transiently transfected, transformed, transduced or infected with a first vector expressing HC polypeptides and a second vector expressing LC polypeptides of an antibody described herein. Such host cells, e.g., mammalian host cells, can express the anti-human LAIR1 antibodies described herein. Mammalian host cells known to be capable of expressing antibodies include CHO cells, HEK293 cells, COS cells, and NS0 cells.

In some embodiments, the cell, e.g., host cell, comprises a vector comprising a first nucleic acid sequence encoding SEQ ID NO: 9 or 25, and a second nucleic acid sequence encoding SEQ ID NO: 10. In some embodiments, the cell, e.g., host cell, comprises a vector comprising a first nucleic acid sequence encoding SEQ ID NO: 21 or 27, and a second nucleic acid sequence encoding SEQ ID NO: 22.

In some embodiments, the cell, e.g., host cell, comprises a first vector comprising a nucleic acid sequence encoding SEQ ID NO: 9 or 25, and a second vector comprising a nucleic acid sequence encoding SEQ ID NO: 10. In some embodiments, the cell, e.g., host cell, comprises a first vector comprising a nucleic acid sequence encoding SEQ ID NO: 21 or 27, and a second vector comprising a nucleic acid sequence encoding SEQ ID NO: 22.

The present disclosure further provides a process for producing an anti-human LAIR1 antibody described herein by culturing the host cell described above, e.g., a mammalian host cell, under conditions such that the antibody is expressed and recovering the expressed antibody from the culture medium. The culture medium, into which an antibody has been secreted, may be purified by conventional techniques. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

Also provided are antibodies produced by any of the processes described herein.

In another aspect, provided herein are pharmaceutical compositions comprising an antibody, nucleic acid, or vector described herein. Such pharmaceutical compositions can also comprise one or more pharmaceutically acceptable excipient, diluent or carrier. Pharmaceutical compositions can be prepared by methods well known in the art (e.g., Remington: The Science and Practice of Pharmacy, 22nd ed. (2012), A. Loyd et al., Pharmaceutical Press).

The anti-human LAIR1 antibodies, nucleic acids, vectors, or pharmaceutical compositions described herein can be used for treating an autoimmune disease or fibrotic disease. Examples of such autoimmune diseases or fibrotic diseases include rheumatoid arthritis, psoriasis, systemic lupus erythematosus, lupus nephritis, pemphigus vulgaris, systemic sclerosis, idiopathic pulmonary fibrosis, scleroderma, scleroderma-associated interstitial lung disease, IgG4 related disease, chronic fibrosing interstitial lung diseases, ulcerative colitis, Crohn's disease, hidradenitis suppurativa, atopic dermatitis, or multiple sclerosis.

In some embodiments, provided herein are methods of treating an autoimmune disease or a fibrotic disease, in a subject (e.g., a human patient) in need thereof, by administering to the subject a therapeutically effective amount of an anti-human LAIR1 antibody, a nucleic acid encoding such an anti-human LAIR1 antibody, a vector comprising such a nucleic acid, or a pharmaceutical composition comprising such an anti-human LAIR1 antibody, nucleic acid or vector, as described herein. The antibodies, nucleic acids, vectors, or pharmaceutical compositions described herein may be administered by parenteral routes (e.g., subcutaneously or intravenously).

Also provided are anti-human LAIR1 antibodies, nucleic acids, vectors, or pharmaceutical compositions described herein for use in therapy. Furthermore, the present disclosure also provides anti-human LAIR1 antibodies, nucleic acids, vectors, or pharmaceutical compositions described herein for use in the treatment of an autoimmune disease or a fibrotic disease.

Provided herein are also use of the anti-human LAIR1 antibodies, nucleic acids, vectors, or pharmaceutical compositions described herein in the manufacture of a medicament for the treatment of an autoimmune disease or a fibrotic disease.

Also provided is an antibody, or antigen fragment thereof, that binds human LAIR1 protein wherein the antibody binds an epitope comprising: one or more amino acid residues selected from FVCRGPVGVQTFRLER (SEQ ID NO: 32) and one more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: two or more amino acid residues selected from FVCRGPVGVQTFRLER (SEQ ID NO: 32) and two or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: three or more amino acid residues selected from FVCRGPVGVQTFRLER (SEQ ID NO: 32) and three or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: four or more amino acid residues selected from FVCRGPVGVQTFRLER (SEQ ID NO: 32) and four or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: five or more amino acid residues selected from FVCRGPVGVQTFRLER (SEQ ID NO: 32) and five or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: six or more amino acid residues selected from FVCRGPVGVQTFRLER (SEQ ID NO: 32) and six or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: seven or more amino acid residues selected from FVCRGPVGVQTFRLER (SEQ ID NO: 32) and seven or more amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the antibody, or antigen fragment thereof, that binds human LAIR1 protein is one wherein the antibody binds an epitope comprising: eight, nine, ten, eleven, twelve, thirteen or fourteen or more amino acid residues selected from FVCRGPVGVQTFRLER (SEQ ID NO: 32) and eight, nine, ten, eleven, twelve, thirteen or fourteen amino acid residues from VSQASPSESEARFRI (SEQ ID NO: 33) wherein the amino acid residues are selected from amino acids 26 to 41 and 53 to 68 wherein the amino acid positions correspond to SEQ ID NO:29.

Preferably, the epitope comprises FVCRGPVGVQTFRLER (SEQ ID NO:32) and VSQASPSESEARFRI (SEQ ID NO:33) wherein the amino acid residues are selected from amino acids 26 to 41 and 53 to 68 wherein the amino acid positions correspond to SEQ ID NO:29.

Also provided is an antibody, or antibody fragment thereof, that binds the epitope as defined above of human LAIR1 protein, wherein the antibody comprises a VH and a VL, wherein the VH comprises HCDR1, HCDR2 and HCDR3, and the VL comprises LCDR1, LCDR2 and LCDR3, wherein HCDR1 comprises SEQ ID NO: 34, HCDR2 comprises SEQ ID NO: 35, HCDR3 comprises SEQ ID NO: 36, LCDR1 comprises SEQ ID NO: 37, LCDR2 comprises SEQ ID NO: 5 and LCDR3 comprises SEQ ID NO: 38. In some embodiments, the anti-human LAIR1 antibody has a human IgG4 isotype. In some embodiments, the anti-human LAIR1 antibody has a modified human IgG4 isotype. In some embodiments, the anti-human LAIR1 has a modified human IgG4 hinge region comprising a S228P mutation (according to the EU Index Numbering), which reduces the IgG4 Fab-arm exchange in vivo (see Labrijn, et al., Nat. Biotechnol. 2009, 27(8):767). In some embodiments, the antibody comprises a heavy chain (HC) comprising SEQ ID NO: 39 and a light chain (LC) comprising SEQ ID NO: 40. In some embodiments, the anti-human LAIR1 antibodies comprise a HC having at least 95% sequence identity to SEQ ID NO: 39 and a LC having at least 95% sequence identity to SEQ ID NO: 40.

Preferably, the antibody, or antigen fragment thereof, that binds human LAIR1 protein is Ab0.

Preferably, the antibody, or antigen fragment thereof, that binds human LAIR1 protein is a human antibody.

Preferably, the antibody, or antigen fragment thereof, that binds the epitope according to the present invention is an antibody that agonizes human LAIR1 protein.

Also provided is an anti-LAIR1 antibody, or antibody fragment thereof, which competes for binding to the epitope of the present invention with any one of the antibodies defined according to the present invention.

Preferably, the anti-LAIR1 antibody, or antigen fragment thereof, which binds the epitope is Ab0.

In some embodiments, the anti-LAIR1 antibody, or antibody fragment thereof, that competes for binding to the epitope with the anti-LAIR1 antibody defined below is Ab1, Ab2, Ab3 or Ab4.

In some embodiments, the anti-LAIR1 antibody is an antibody, or antibody fragment thereof, that binds the epitope of human LAIR1 protein, as defined above, wherein the antibody comprises a VH and a VL, wherein the VH comprises HCDR1, HCDR2 and HCDR3, and the VL comprises LCDR1, LCDR2 and LCDR3, wherein HCDR1 comprises SEQ ID NO: 34, HCDR2 comprises SEQ ID NO: 35, HCDR3 comprises SEQ ID NO: 36, LCDR1 comprises SEQ ID NO: 37, LCDR2 comprises SEQ ID NO: 5 and LCDR3 comprises SEQ ID NO: 38. In some embodiments, the anti-human LAIR1 antibody has a human IgG4 isotype. In some embodiments, the anti-human LAIR1 has a modified human IgG4 hinge region comprising a S228P mutation (according to the EU Index Numbering), which reduces the IgG4 Fab-arm exchange in vivo (see Labrijn, et al., Nat. Biotechnol. 2009, 27(8):767). In some embodiments, the antibody comprises a heavy chain (HC) comprising SEQ ID NO: 39 and a light chain (LC) comprising SEQ ID NO: 40. In some embodiments, the anti-human LAIR1 antibodies comprise a HC having at least 95% sequence identity to SEQ ID NO: 39 and a LC having at least 95% sequence identity to SEQ ID NO: 40.

The epitope is preferably determined by the hydrogen-deuterium exchange (HDX) mapping technique.

An advantage of the anti-human LAIR1 antibody, or antigen fragment thereof, is that it is able to turn on the body's natural immune inhibitory mechanisms. This may lead to both target-cell-specific efficacy and key safety benefits over current immunomodulatory therapies.

The anti-human LAIR1 antibody of the present invention preferably has one or more of the following key properties:
binding to human LAIR1 and cross-reactivity to cynomolgus monkey LAIR1
binding to a unique epitope (non-ligand blocking).
demonstrates agonism of LAIR1.
antibody-mediated agonism leading to attenuation of human primary B cell activation in a dose dependent manner
demonstrates in vivo efficacy in a humanized mouse model of graft vs. host disease.
preclinical efficacy in mouse model of lupus nephritis

As used herein, the term “a,” “an,” “the” and similar terms used in the context of the present disclosure (especially in the context of the claims) are to be construed to cover both the singular and plural unless otherwise indicated herein or clearly contradicted by the context.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100 to 125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). The North CDR definitions are used for the anti-human LAIR1 antibodies described herein.

The present disclosure also includes antibody fragments or antigen-binding fragments, which comprise at least a portion of an antibody retaining the ability to specifically interact with an antigen such as Fab, Fab′, F(ab′)2, Fv fragments, scFv, scFab, disulfide-linked Fvs (sdFv), a Fd fragment and linear antibodies.

The term “epitope” refers to the amino acid residues, of an antigen, that are bound by an antibody. An epitope may be a linear epitope, a conformational epitope, or a hybrid epitope.

The term “epitope” may be used in reference to a structural epitope. A structural epitope, according to some embodiments, may be used to describe the region of an antigen which is covered by an antibody (e.g., an antibody's footprint when bound to the antigen).

An epitope can be determined according to different experimental techniques, also called “epitope mapping techniques”. It is understood that the determination of an epitope may vary based on the different epitope mapping techniques used and may also vary with the different experimental conditions uses e.g., due to the conformation changes or cleavages of the antibody induced by specific experimental conditions. Epitope mapping techniques are known in the art (e.g. Rockberg and Nivebrant, Epitope mapping Protocols: Methods in Molecular Biology, Humana press, 3rd ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

As used herein, the term “competes for binding” or “competes with”, refers to two antibodies which cross-compete (i.e., compete against each other) for binding to the same antigen. In some embodiments, two antibodies may compete for binding to the same antigen where they bind to spatially overlapping regions of the same antigen. In some embodiments, two antibodies may compete for binding to the same antigen where the antibodies bind to non-overlapping regions of the antigen, but the binding of one antibody blocks binding by the other antibody, for example, due to steric hindrance or conformational changes of the antigen induced by the first antibody. Numerous types of competitive binding assays can be used to determine if one antibody competes with another, for example, solid phase direct or indirect radioimmunoassay (MA), solid phase direct or indirect enzyme immunoassay (EIA), sandwich competition assay, surface plasmon resonance, bio-layer interferometry, or flow cytometric methodology. Epitope binning may be carried out using Carterra technology (e.g. PLoS One, 2014 Mar. 20; doi: 10.137/j ournal.pone. 0092451, Y. Abdiche et al.).

The term “agonist” or “agonistic”, as used herein, refers to an antibody or antibody fragment that is capable of inducing or increasing one or more activities or functions associated with human LAIR1, e.g., one or more activities or functions associated with human LAIR1 described in the Examples.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

An “effective amount” refers to an amount necessary (for periods of time and for the means of administration) to achieve the desired therapeutic result. An effective amount of an antibody may vary according to factors such as the disease state, age, sex, and weight of the individual, and the ability of the antibody to elicit a desired response in the individual. An effective amount is also one in which any toxic or detrimental effects of the antibody are outweighed by the therapeutically beneficial effects.

The term “Fc region” as used herein refers to a region of an antibody, which comprises the CH2 and CH3 domains of the antibody heavy chain. Optionally, the Fc region may include a portion of the hinge region or the entire hinge region of the antibody heavy chain.

The term “LAIR1” as used herein, unless stated otherwise, refers to human leukocyte associated immunoglobulin like receptor 1 (also known as CD305). The amino acid sequence of human LAIR1 isoform a (longest isoform) can be found at NCBI Accession No. NP 002278.2:

(SEQ ID NO: 29)
MSPHPTALLGLVLCLAQTIHTQEEDLPRPSISAEPGTVIPLGSHVTFVCR
GPVGVQTFRLERDSRSTYNDTEDVSQASPSESEARFRIDSVREGNAGLYR
CIYYKPPKWSEQSDYLELLVKESSGGPDSPDTEPGSSAGPTQRPSDNSHN
EHAPASQGLKAEHLYILIGVSVVFLFCLLLLVLFCLHRQNQIKQGPPRSK
DEEQKPQQRPDLAVDVLERTADKATVNGLPEKDRETDTSALAAGSSQEVT
YAQLDHWALTQRTARAVSPQSTKPMAESITYAAVARH

Several shorter isoforms of human LAIR1 have been reported, including isoform b (NCBI Accession No. NP 068352.2), isoform c (NCBI Accession No. NP_001275952.2), isoform e (NCBI Accession No. NP_001275954.2), isoform f (NCBI Accession No. NP_001275955.2), isoform g (NCBI Accession No. NP_001275956.2). The term “LAIR1” is used herein to refer collectively to all known human LAIR1 isoforms.

The amino acid sequence of cynomolgus monkey LAIR1 can be found at) CP 045236925.1 (isoform X1) XP 045236926.1 (isoform X2), XP 045236927.1 (isoform X3), or XP 045236928.1 (isoform X4).

The terms “nucleic acid” or “polynucleotide”, as used interchangeably herein, refer to polymers of nucleotides, including single-stranded and/or double-stranded nucleotide-containing molecules, such as DNA, cDNA and RNA molecules, incorporating native, modified, and/or analogs of, nucleotides.

The term “subject”, as used herein, refers to a mammal, including, but are not limited to, a human, chimpanzee, ape, monkey, cattle, horse, sheep, goat, swine, rabbit, dog, cat, rat, mouse, guinea pig, and the like. Preferably the subject is a human.

As used herein, “treatment” or “treating” refers to all processes wherein there may be a slowing, controlling, delaying or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

The following examples are offered to illustrate, but not to limit, the claimed invention.

### Example 1. Generation of the Anti-Human LAIR1 Antibodies

Human anti-human LAIR1 antibodies were generated using AlivaMab® human transgenic mice and the cloning of anti-LAIR1 variable regions. Mice were immunized with human LAIR1 fused to human Fc with a His tag and a TEV cleavage site (SEQ ID NO: 30), with or without co-administration of human LAIR2 fused to human Fc with a TEV cleavage site (SEQ ID NO:31), using standard procedures and antigen-specific B cells were isolated by standard sorting methods using fluorophore-labeled LAIR1. The LAIR1 immunogen has the following amino acid sequence:

(SEQ ID NO: 30)
QEEDLPRPSISAEPGTVIPLGSHVTFVCRGPVGVQTFRLERESRSTYNDT
EDVSQASPSESEARFRIDSVSEGNAGPYRCIYYKPPKWSEQSDYLELLVK
ETSGGPDSPDTEPGSSAGPTQRPSDNSHNEHAPASQGLKAEHENLYFQGE
PKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGKHHHHHH.

The LAIR2 immunogen has the following amino acid sequence:

(SEQ ID NO: 31)
QEGALPRPSISAEPGTVISPGSHVTFMCRGPVGVQTFRLEREDRAKYKDS
YNVFRLGPSESEARFHIDSVSEGNAGLYRCLYYKPPGWSEHSDFLELLVK
ESSGGPDSPDTEPGSSAGTVPGTEASGFDAPENLYFQGEPKSSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK

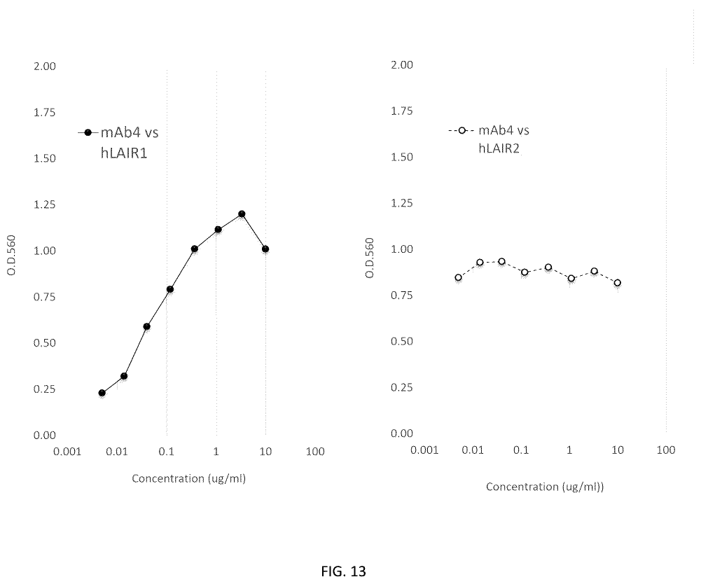

The variable regions of the LAIR1 specific antibodies were cloned, expressed and the activity of the recombinant antibodies confirmed by ELISA, and demonstrated selectivity for LAIR1 and no activity against LAIR2 (FIG. 13). The antibodies were then engineered for higher affinity following mutagenesis of the CDR residues and identifying enhancing mutations from combined residues for each CDR. Individual combinatorial clones were sequenced. The heavy chain and light chain CDRs, VH/VL, and HC/LC sequences of exemplary anti-human LAIR1 antibodies are provided in Table 1.

Anti-human LAIR1 antibodies can be generated by recombinant DNA technology. Such antibodies can be expressed in a mammalian cell line such as HEK293 or CHO, either transiently or stably transfected with an expression system using an optimal predetermined HC:LC vector ratio or a single vector system encoding both HC and LC. Clarified media, into which the antibody has been secreted, can be purified using the commonly known techniques.

### Example 2: Characterization of the Anti-Human LAIR1 Antibodies

### Antibody Binding Affinity and Kinetics

Antibody binding affinity and kinetics were determined by surface plasmon resonance (SPR) using a Biacore 8K (Cytivia Life Sciences). Measurements were performed at 37° C. using HBS-EP+ as the running buffer (150 mM sodium chloride, 3 mM EDTA, 0.05% (w/v) surfactant P-20, and 10 mM HEPES, pH7.4). Binding experiments used the soluble, extracellular domain (ECD) of LAIR1 (SEQ ID NO: 17), produced recombinantly, which was diluted to working concentration in HBS-EP+ containing 0.1 mg/mL Bovine serum albumin. Goat anti-Human kappa (Southern Biotech) was immobilized on all eight flow cells of a CM4 sensor chip using an amine coupling kit.

The soluble, extracellular domain (ECD) of LAIR1 has the following amino acid sequence:

(SEQ ID NO: 17)
QEEDLPRPSISAEPGTVIPLGSHVTFVCRGPVGVQTFRLERESRSTYNDT
EDVSQASPSESEARFRIDSVSEGNAGPYRCIYYKPPKWSEQSDYLELLVK
ETSGGPDSPDTPGSSAGPTQRPSDNSHNEHAPASQGLKAEHENLYFQ.

Binding was evaluated using multiple analytical cycles. Each cycle is performed at a flow rate of 30 μL/minute and consists of the following steps: injection of antibody over a different flow cell (25 μL of antibody at 0.5 μg/mL at 10 μL/minute), injection of 75 μL (30 μL/min, for 150 seconds) of each LAIR1-ECD dilution (starting at 1 μM and using three-fold serial dilutions to 1.4 nM for each cycle, with one injection for each concentration) followed by a 1200 second delay for dissociation, and chip surface regeneration using three 15 μL injections (30 μL/min, for 30 seconds) of 10 mM glycine hydrochloride, pH1.7. Association and dissociation rates for each cycle were determined by fitting of the biosensor data to a simple 1:1 association model using the provided instrument analysis software to extract the kₒₙ and k_{off} rate constants; the equilibrium binding constant K_{D} was calculated using the relationship K_{d}=k_{off}/kₒₙ.

Table 2 shows the binding affinity and kinetics of the anti-human LAIR1 mAbs.

TABLE 2
Binding affinity and kinetics of anti-human
LAIR1 mAbs determined by Biacore at 37° C.

| Antibody ID | kₒₙ (M⁻¹ s⁻¹) | k_{off} (sec⁻¹) | K_{D} (nM) |
|---|---|---|---|
| mAb1 | 1.44 × 10⁶ | 1.74 × 10⁻⁴ | 0.121 |
| mAb2 | 1.09 × 10⁶ | 3.41 × 10⁻⁴ | 0.312 |
| mAb3 | 1.34 × 10⁶ | 1.68 × 10⁻⁴ | 0.126 |
| mAb4 | 1.09 × 10⁶ | 3.17 × 10⁻⁴ | 0.291 |

### Physical-Chemical Properties of Exemplified LAIR1 Antibodies

### Thermal Stability:

Differential Scanning calorimetry (DSC) was used to evaluate the stability of the exemplified LAIR1 antibodies against thermal denaturation. DSC was run using a Malvern MircoCal VP-DSC instrument. Samples in PBS buffer were heated from 20° C. to 110° C. at a constant rate of 60° C./hour. Analysis methods were performed using the MicroCal VP-Capillary DSC Automated Analysis program. Baseline corrections were performed, and Tm onset and TMs were determined. The results as demonstrated in Table 3, showed the exemplified LAIR1 antibodies had Tm onset>60° C. and were thermal stable.

TABLE 3
Summary of DSC results

| Sample | Tm onset (° C.) | Tm₁(° C.) | Tm₂(° C.) |
|---|---|---|---|
| mAb1 | 65.1 | 72.4 | 76.5 |
| mAb2 | 63.5 | 70.3 | 80.8 |
| mAb3 | 61.9 | 69.2 | 75.9 |
| mAb4 | 61.1 | 68.8 | 80.3 |

### Solubility:

Sufficiently high solubility is desired to enable convenient dosing. In addition, maintaining the antibody in monomeric state without high molecular weight (HMW) aggregation at high concentration is also desirable. Solubility of the exemplified LAIR1 antibodies is analyzed by concentrating 15 mg of an exemplified antibody with a 10 K molecular weight cut-off filter (Amicon U.C. filters, Millipore, catalog #UFC903024) to a volume of less than 100 μl. The final concentration of the sample was measured by SoloVPE spectrophotometer (C Technologies, INC). The Following procedures substantially as described above, the exemplified antibodies display a solubility of greater than 150 mg/mL (at 5 mM histidine, pH 6.0) and 200 mg/ml (at pH 7.4 in PBS buffer). The concentrated solution samples are stored at 4° C. for 1 week, followed by 1 week storage at −5° C. The aggregation profiles of the antibodies after two-week storage are assessed using size exclusion chromatography (SEC). The results as demonstrated in Table 4, only low levels of high molecular weight (HMW) aggregates (around 1%) are present at high concentration and no phase separation is observed.

TABLE 4
Summary of solubility results

| | 5 mM histidine, pH 6 | | PBS | |
|---|---|---|---|---|
| | Concentration | % HMW | Concentration | % HMW |
| Samples | (mg/mL) | (2 w) | (mg/mL) | (2 w) |
| mAb1 | 167 | 1 | 211 | 1 |
| mAb2 | 158 | 0.8 | 198 | 1 |
| mAb3 | 157 | 0.9 | 234 | 1 |
| mAb4 | 161 | 1.1 | 210 | 1 |

### Photostability:

Photostability of the exemplified antibodies were assessed at a high concentration (approximately 100 mg/mL) in 5 mM histidine buffer (pH 6.0) with excipients. The concentrated samples were exposed to 20% ICH Q1B minimum exposure of 40-watt hr/m² UV light (4 hours at 10 watt/m²)+240 klux hours (8 klux for 30 hours) at 25° C. in the photo chamber. The protected concentrated samples (wrapped in aluminum foil) were used as dark controls and placed alongside the authentic samples. Upon exposure, samples were analyzed for the percentage of UMW aggregates growth (A % UMW) with SEC. Results provided in Table 5 demonstrate that after expose to 20% ICH guidelines, the exemplified antibodies have a percentage of HMW growth between 5 to 7%.

TABLE 5
Summary of % HMW growth upon photo exposure

| | Δ% HMW upon photo exposure (relative |
|---|---|
| Sample | to dark control) |
| mAb1 | 7 |
| mAb2 | 6.7 |
| mAb3 | 5.8 |
| mAb4 | 5 |

### Chemical Stability:

Chemical stability facilitates the development of drug formulations with sufficient shelf-life. Chemical stability of the exemplified antibodies is assessed by formulating the exemplified antibodies to a concentration of 100 mg/ml in a buffered solution, pH 6. Formulated samples are incubated for four weeks at 4° C., and 35° C. in an accelerated degradation study. Changes in fragmentation and aggregation profile of the antibody are assessed using capillary electrophoresis sodium dodecyl sulfate (CE-SDS) and SEC according to standard procedures. Following procedures substantially as described above, the exemplified antibodies demonstrate chemical stability results presented in Table 6.

TABLE 6
Summary of Δ% fragments measured by CE-SDS
and Δ% HMW measured by SEC over four weeks
at 35° C., relative to samples incubated at 4° C.

| | Δ% Fragments after 4 weeks | Δ% HMW after 4 weeks at |
|---|---|---|
| Sample | at 35° C. (relative to 4° C.) | 35° C. (relative to 4° C.) |
| mAb1 | 0.4 | 2.5 |
| mAb2 | 0.2 | 1 |
| mAb3 | 1.3 | 2.5 |
| mAb4 | 0.5 | 0.9 |

Results provided in Table 6 demonstrate that after 4 weeks storage at 35° C., the exemplified antibodies have a percentage of fragments increase between 0.2 to 1.3%. The levels of HMW growth of the exemplified antibodies are between 0.9 to 2.5%.

The chemical stability data indicate that the exemplified antibodies have sufficient chemical stability to facilitate development of solution formulations with adequate shelf life.

In summary, the exemplified LAIR1 antibodies demonstrate good solubility, low aggregation, chemical stability and physical stability characteristics essential for parental therapeutic administration.

### Example 3: Immunogenicity Assessment of the Anti-Human LAIR1 Antibodies

A panel of in vitro and ex vivo methods were used to characterize the relative risk of immunogenicity of the exemplified LAIR1 mAbs as described below.

### Dendritic Cell (DC) Internalization Assay:

This assay was performed to investigate the internalization of molecules by CD14+ monocytes derived dendritic cells. CD14+ monocytes were isolated from periphery blood mononuclear cells (PBMCs) and were cultured and differentiated into DC following standard protocols (See Wen, Y., et al., AAPS J 2020 Apr. 16; 22(3):68). Briefly, PBMCs were isolated using density-gradient centrifugation with Ficoll (#17-1440-02, GE Healthcare) and Sepmate 50 (#15450, STEMCELL Technologies) from LRS-WBC. CD14+ monocytes were isolated using positive selection with a CD14+ microbead kit (#130-050-201, Miltenyi Biotec) following the manufacturer's manual. Cells were then cultured at 1 million/mL with 1000 unit/mL GM-CSF and 600 unit/mL IL-4 for 6 days to drive to immature dendritic cells (MDDC) in RPMI medium with L-glutamine and 25 mM HEPES supplemented with 10% FBS, 1 mM sodium pyruvate, 1× penicillin-streptomycin, 1× non-essential amino acids, and 55 μM 2-mercaptoethanol (hereafter referred to complete RPMI medium or medium, purchased from Life Technologies). The medium was changed twice, on day 2 and day 5. On day 6, cells were gently collected with a cell scraper and used for experiment. To obtain mature DCs, cells were treated with 1 μg/mL LPS for 4 hours.

Individual test molecules were normalized to 1 mg/mL with PBS and then further diluted to 8 μg/mL in complete RPMI medium. The detection prob, Fab-TAMRA-QSY7, was diluted to 5.33 μg/mL in complete RPMI medium. The antibody and Fab-TAMRA-QSY7 were mixed with equal volume and incubated for 30 min at 4° C. in dark for complex formation. MDDC were resuspended at 4 million/mL in complete RPMI medium and seeded at 50 μL per well in a 96-well round-bottom plate, to which 50 μL of the antibody/probe complex was added. Cells were incubated for 24 hours at 37° C. in a CO2 incubator. Cells were washed with 2% FBS PBS and resuspended in 100 μL 2% FBS PBS with Cytox Green live/dead dye. Data were collected on a BD LSR Fortessa X-20 and analyzed in FlowJo. Live single cells were gated and percent of TAMRA fluorescence positive cells was recorded as the readout. To allow the comparison of molecules with data generated from different donors, a normalized internalization index was used. The internalization signal was normalized to IgG1 isotype (normalized internalization index=0) and an internal positive control PC (normalized internalization index=100) using the formula:

$$100 \times \frac{{X}_{TAMRA} - IgG 1 {isotype}_{TAMRA}}{P {C}_{TAMRA} - IgG 1 {isotype}_{TAMRA}}$$

where XTAMRA, IgG1 isotypeTAMRA, and PCTAMRA were the percent of TAMRA-positive population for the test molecule X, IgG1 isotype, and PC respectively. For the normalized internalization index, 0-15 is considered low, >15-30 is considered low to moderate, >30-60 is considered moderate, and >60 is considered high risk of immunogenicity.

TABLE 7
Dendritic Cell Internalization Results
for the Exemplified LAIR1 Antibodies

| Exemplified Antibody | Internalization Index |
|---|---|
| mAb1 | 43.0 |
| mAb2 | 38.0 |
| mAb3 | 33.7 |
| mAb4 | 27.5 |

As shown in Table 7, the exemplified anti-human LAIR1 antibodies have low-to-moderate and moderate risk from the dendritic cell internalization assay.

### MAPPs Assay (MHC-Associated Peptide Proteomics):

MAPPs profiles the human leukocyte antigen class II (HLA-II) presented peptides on human dendritic cells previously treated with test molecule. Primary human dendritic cells from a panel of 10 normal human donors were prepared from buffy coats by isolation of CD-14 positive cells and differentiated into immature dendritic cells by incubation with 20 ng/ml IL-4 and 40 ng/ml GM-CSF in complete RPMI media containing 5% Serum Replacement (Thermo Fisher Scientific, cat #A2596101) for 3 days at 37° C. and 5% CO2 as described (Knierman et al., Cell Rep 2020 Dec. 1; 33(9):108454). Three micromolar of test antibody was added to approximately 5×106 cells on day 4 and fresh media containing 5 μg/ml of LPS to transform the cells into mature dendritic cells was exchanged after 5-hour incubation. The matured cells were lysed in lmL of RIPA buffer with protease inhibitors and DNAse the following day. The lysates were stored at −80° C. until sample analysis.

An automated liquid handling system was used to isolate the HLA-II molecules from thawed lysate using biotinylated anti-pan HLA class II antibody (clone Tu39). The bound receptor-peptide complex was eluted with 5% acetic acid, 0.1% TFA. The eluted HLA-II peptides were passed over a prewashed 10k MWCO filter to remove high molecular weight proteins. The isolated HLA-II peptides were analyzed by nano LC/MS using a Thermo easy 1200 nLC-HPLC system with a Thermo LUMOS mass spectrometer. The separation used a 75 μm×7 cm YMC-ODS C18 column for 65-minute gradient with a 250 nL/min flow rate and 0.1% formic acid in water as A solvent and 80% acetonitrile with 0.1% formic acid as B solvent. Mass spectrometry was run in full scan mode with 240,000 resolution followed by a 3 second data dependent MS/MS cycle comprised of ion trap rapid scans with HCD and EThcD fragmentation.

Peptide identifications were generated by an internal proteomics pipeline (Higgs et al., Methods Mol Biol. 2008; 428:209-30) using multiple search algorithms with no enzyme search parameter against a bovine/human database containing the test molecule sequence. Peptides identified from the test molecules were aligned against the parent sequence. A summary was created for all test molecules that annotates the percent of donors that display peptides with non-germline residues and the number of different regions of the test molecule that display peptides with non-germline residues. Increases in the extent of display of non-germline peptides is associated with increased risk for immunogenicity.

TABLE 8
MAPPs Results for the Exemplified LAIR Antibodies

| | Number regions | Percentage |
|---|---|---|
| | with non- | of donors |
| Exemplified | germline residues | displaying non- |
| Antibody | across all donors | germline regions |
| mAb1 | 3 | 60% |
| mAb2 | 2 | 80% |
| mAb3 | 3 | 60% |
| mAb4 | 2 | 80% |

As shown in Table 8, the exemplified anti-LAIR1 antibodies mAb1 to 4 have moderate risk from the MAPPs assay.

### T Cell Proliferation Assay

This assay assesses the ability of test molecule to activate CD4+ T cells by inducing cellular proliferation (see Walsh, R. E., et al., MAbs. 2020; 12(1): 1764829). Cryopreserved PBMC's were used from 10 healthy donors and the CD8+ T cells were depleted from the PBMC's and labeled with 1 μM Carboxyfluorescein Diacetate Succinimidyl Ester (CFSE). PBMCs were seeded at 4×106 cells/ml/well in AIM-V media (Life Technologies, cat #12055-083) containing 5% CTS™ Immune Cell SR (Gibco, cat #A2596101) and tested in triplicate in 2.0 mL containing the different test molecule, DMSO control, media control, keyhole limpet haemocyanin (KLH; positive control). Cells were cultured and incubated for 7 days at 37° C. with 5% CO2. On day 7, samples were stained with the following cell surface markers: anti-CD3, anti-CD4, anti-CD14, anti-CD19, and DAPI for viability detection by flow cytometry using a BD LSRFortessa™, equipped with a High Throughput Sampler (HTS). Data was analyzed using FlowJo® Software (FlowJo, LLC, TreeStar) and a Cellular Division Index (CDI) was calculated. Briefly, the CDI for each test molecule was calculated by dividing the percent of proliferating CFSEdimCD4+ T cells from molecule-stimulated wells by the percent of proliferating CFSEdimCD4+ T cells in the unstimulated wells. A CDI of >2.5 was considered to represent a positive response. A percent donor frequency across all donors was evaluated. Proliferation in <30% donors is considered low, 30-40% moderate, and >40% high risk for immunogenicity.

TABLE 9
T cell Proliferation Results for the Exemplified LAIR1 Antibodies

| Exemplified | % Positive | Number of positive |
|---|---|---|
| Antibody | Donors | donors (CDI > 2.5) |
| mAb2 | 0% | 0/9 |
| mAb3 | 0% | 0/9 |
| mAb4 | 0% | 0/9 |

As shown in Table 9, the exemplified anti-human LAIR1 antibodies mAb2 to 4 have low risk from the T cell proliferation assay. Anti-human LAIR1 mAb1 was not tested.

### Pre-Existing Reactivity Assay:

This assay was performed to investigate the presence of reactivity derived from pre-existing anti-drug antibodies (PEA), and potentially other cross-reactive proteins, in treatment naïve normal human serum (See Bivi, N., et al., MAbs. 2019 July; 11(5):861-869). Diluted serum from a panel of at least 50 treatment naïve donors was captured overnight on a plate coated with biotinylated test molecule. On the following day, the captured reactive proteins are acid eluted, and then neutralized in the presence of biotinylated and ruthenylated test molecule. If anti-drug antibodies are present, they will bridge the labeled test candidate and form a complex. The complexes are captured by a streptavidin-coated Mesoscale plate, and the resulting signal is referred to as Tier 1 signal (expressed as electrochemiluminescence). This signal is confirmed in Tier 2 by adding excess unlabeled test molecule in the detection step, which results in the suppression of the Tier 1 signal. The presence of pre-existing anti-drug antibodies is expressed as the 90th percentile of Tier 2 inhibition. Results <30% are low, 30%-55% moderate, and >55% high risk for immunogenicity.

TABLE 10
Pre-Existing Reactivity Results for
the Exemplified LAIR1 Antibodies

| Exemplified Antibody | 90th Percentile T2 Inhbition |
|---|---|
| mAb1 | 7.1% |
| mAb2 | 7.6% |
| mAb3 | 8.0% |
| mAb4 | 25.8% |

As shown in Table 10, the exemplified anti-human LAIR1 antibodies mAb1 to 4 have low risk from the pre-existing reactivity assay.

### Example 4: In Vitro Activities of the Anti-Human LAIR1 Antibodies

### Cell-Based Binding:

The anti-human LAIR1 antibodies were evaluated for binding to human and cynomolgus monkey (cyno) LAIR1 engineered cell lines and primary human T cells that endogenously express LAIR1. Jurkat-hLAIR1+ (Jurkat cell overexpressing human LAIR1), Jurkat-LAIR1ko (Jurkat cell knocking out human LAIR1), Jurkat-cyLAIR1+ cells (Jurkat LAIR1ko cell expressing cyno LAIR1), and primary human T cells were incubated with the anti-human LAIR1 test antibodies. Serial dilutions of antibodies ranging 0.0017 μg/mL-3.33 μg/mL were incubated with the cells for 20 minutes at 4° C. Cells were then washed and incubated with anti-human IgG Alexa Fluor 647 secondary antibody for 20 minutes at 4° C. Cells were then washed and antibody binding was evaluated by flow cytometry. For primary human T cells, cells were also stained for CD4 and CD8 to delineate CD4+ and CD8+ T cells.

As shown in Table 11, all anti-human LAIR1 test antibodies bound with similar binding intensity to Jurkat-hLAIR1+ cells, primary human CD4+ and CD8+ T cells. The EC50 of binding to Jurkat-cyLAIR1+ cells were within 2-fold of EC50 of binding to Jurkat-hLAIR1+ cells. The test anti-LAIR1 antibodies demonstrated no binding to LAIR1ko, which is the control cell line that does not express LAIR1.

TABLE 11
EC50 of the anti-human LAIR1 antibody binding to Jurkat-hLAIR1+, Jurkat-
cyLAIR1+, primary human CD4+ T cells, and primary human
CD8+ T cells. (Data are representative of 1-5 independent
experiments; 2 donors evaluated for primary T cell binding.)

| | | | Primary Human | Primary Human |
|---|---|---|---|---|
| | Jurkat-hLAIR1+ | Jurkat-cyLAIR1+ | CD4+ T cells | CD8+ T cells |
| Antibody ID | (ng/mL) | (ng/mL) | (ng/mL) | (ng/mL) |
| mAb1 | 54.75 | 78.44 | 36.99 | 48.87 |
| mAb3 | 53.43 | 83.38 | 40.1 | 53.23 |
| mAb2 | 58.01 | 82.39 | 28.82 | 41.4 |
| mAb4 | 56.53 | 89.34 | 16.24 | 34.9 |

### Jurkat-NFAT Activation:

The effects of anti-human LAIR1 antibodies on Jurkat cell NFAT activation were evaluated. Jurkat-NFAT-Luciferase cells expressing human LAIR1 (Jurkat-hLAIR1+), cyno LAIR1 (Jurkat-cyLAIR1+) or LAIR1ko-deficient (Jurkat-LAIR1ko) were TCR-stimulated with anti-human CD3 antibody in the presence of anti-human LAIR1 antibodies. Specifically, CHO-K1 cells were seeded overnight in 96-well flat bottom tissue-culture sterile plates. When confluency reached 85-95%, cells were washed with RPMI/5% human serum and incubated with anti-human CD3 antibody at 10 μg/mL for 1 hour at 37° C. Unbound CD3 antibody was then removed, cells were washed, and incubated with anti-human LAIR1 antibodies at the indicated concentrations for 20 minutes at 37° C. 1×105 Jurkat-hLAIR1+, Jurkat-cyLAIR1+, or Jurkat-LAIR1ko cells were added and incubated for 6 hours at 37° C. Jurkat cells were then transferred to opaque, flat, clear bottom 96-well plates and equal volume BrightGlo luciferase was added. Following 2 minute incubation for lysis, NFAT activity (via luciferase readout) was evaluated by luminometer. The test anti-LAIR1 antibodies were evaluated at concentrations ranging 0.1 ng/mL-1 μg/mL, serial dilution with 3-fold titration down for 8 steps, in activation assay.

As shown in Table 12, all anti-human LAIR1 test antibodies inhibited Jurkat NFAT activation in both human and cyno LAIR1 expressing Jurkat-NFAT cells, with inhibition ranging 60-70%, whereas isotype controls had no impact on NFAT activity. Similar IC50 values were observed among the test anti-human LAIR1 antibodies (Table 12). No IC50 values were available for Jurkat-cyLAIR1+ cells. Anti-human LAIR1 antibodies had no effect on NFAT activity in Jurkat-LAIR1ko cells. No values shown for Jurkat-LAIR1ko as the anti-LAIR1 antibodies demonstrated no inhibitory effect to this control cell line.

TABLE 12
In vitro agonistic activity of the anti-human LAIR1 antibodies
on NFAT activity of Jurkat cells following TCR stimulation.
(Data are representative of 2 to 5 independent experiments.)

| | Inhibition of NFAT Activity | | IC₅₀ of antibody |
|---|---|---|---|
| | (% inhibition vs. no antibody) | | activity (ng/mL) |
| Antibody ID | Jurkat-hLAIR1+ | Jurkat-cyLAIR1+ | Jurkat-hLAIR1+ |
| mAb1 | 54.22 | 44.21 | 9.42 |
| mAb3 | 60.28 | 67.09 | 11.92 |
| mAb2 | 49.81 | 59.56 | 8.578 |
| mAb4 | 60.35 | 65.54 | 6.661 |

Anti-Human LAIR1 Antibody, mAb4, Inhibits NFAT Activation in an In Vitro Cell Based Agonism Assay:

The ability of a LAIR1 agonist antibody to inhibit NFAT activation in a human T cell line overexpressing human LAIR1 was determined as follows.

Jurkat-NFAT-luciferase reporter cells were engineered to overexpress human LAIR1 (Jurkat-hLAIR1+) via lentiviral transduction. Jurkat-hLAIR1+ cells were TCR-stimulated with anti-human CD3 antibody (clone OKT3) in the presence of cross-linked anti-human LAIR1 mAb4 or hIgG4SP isotype control antibody. Antibodies were cross-linked using a Chinese Hamster Ovary (CHO) cell line engineered to express human Fc gamma RIIb.

CHO-K1 cells were seeded overnight 37° C. in 96-well flat bottom tissue-culture sterile plates. When confluency reached 85-95%, cells were washed with RPMI/5% human serum and incubated with anti-human CD3 antibody for 1 hour at 37° C. Unbound CD3 antibody was then removed, cells were washed, and incubated with mAb4 or hIgG4SP isotype antibodies for 20 minutes at 37° C. 1×105. Jurkat-hLAIR1+ cells were added to plates and incubated for 6 hours at 37° C. Jurkat-hLAIR1+ cells were then transferred to opaque, flat, clear bottom 96-well plates and equal volume BrightGlo luciferase was added. Following 2 minutes of incubation for cell lysis, NFAT activity (via luciferase readout) was evaluated by luminometer. Antibodies were evaluated at concentrations ranging 0.001-6.7 nM (0.2-1000 ng/mL), serial dilution for IC50 evaluation of NFAT activity.

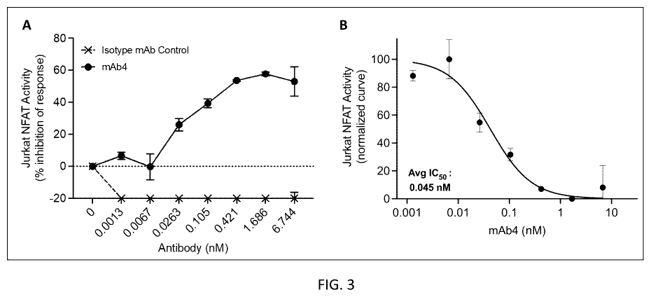

As shown in FIG. 3, mAb4 inhibited Jurkat-hLAIR1+ NFAT activation, with inhibition ranging 60-70% (calculated as % NFAT activity with antibody vs. no antibody) and IC50 value 0.045 nM (6.7 ng/mL), whereas hIgG4SP isotype control antibody had no impact on NFAT activity. As a parallel control, antibodies were evaluated in LAIR1-deficient Jurkat-NFAT-luciferase cells (Jurkat-hLAIR1ko). mAb4 had no effect on NFAT activity in Jurkat-hLAIR1ko cells (data not shown).

### Primary Human B Cells:

The effects of anti-human LAIR1 antibodies on primary human B cell cytokine response were evaluated. Primary human B cells were BCR-stimulated with anti-human IgM antibody plus IL4 in the presence of the test anti-human LAIR1 antibodies at concentrations ranging 0.00128 ng/mL-8 ng/mL. Specifically, CHO-K1 cells were seeded overnight in 96-well flat bottom tissue-culture sterile plates. When confluency reached 85-95%, cells were washed with RPMI/5% human serum and incubated with anti-human LAIR1 antibodies at indicated concentrations for 20 minutes at 37° C. 1-1.5×105 B cells were added and incubated room temperature for an additional 20 minutes to allow for cell/antibody interaction. Stimulant or control was then added (20 ng/mL IL-4+5 μg/mL anti-human IgM or media alone as non-stimulation control) and cells were incubated for 72 hours at 37° C. Test antibodies were also evaluated against anti-human IgG isotype controls. The effects of LAIR1 engagement on B cell IL-6 response was evaluated at 72 hours by ELISA and reported as % inhibition compared to no antibody control.

As shown in Table 13, all anti-human LAIR1 test antibodies inhibited IL6 response, with inhibition at top dose 8 ng/mL ranging 20-70% depending on donor, whereas isotype controls had no impact on cytokine response.

TABLE 13
In vitro agonistic activity of anti-human LAIR1 antibodies
on primary B cells following BCR stimulation.

| | Inhibition of IL-6 response |
|---|---|
| | at 8 ng/mL |
| Antibody ID | (% inhibition vs. no antibody) |
| mAb1 | 60.2 |
| mAb3 | 69.57 |
| mAb2 | 35.41 |
| mAb4 | 42.66 |

Anti-Human LAIR1 Antibody, mAb4, Inhibits Primary B Cell Cytokine Response in an In Vitro Cell Based Agonism Assay:

The ability of a LAIR1 agonist antibody to inhibit BCR-stimulation induced IL-6 response in primary human B cells was determined as follows.

Primary human B cells (n=6 donors) were BCR-stimulated with anti-human IgM antibody plus IL-4 in the presence of cross-linked anti-human LAIR1 mAb4 or hIgG4SP isotype control antibody. Antibodies were cross-linked using a Chinese Hamster Ovary (CHO) cell line engineered to express human Fc gamma Rub.

CHO-K1 cells were seeded overnight 37° C. in 96-well flat bottom tissue-culture sterile plates. When confluency reached 85-95%, cells were washed with RPMI/5% human serum and incubated with mAb4 or hIgG4SP isotype antibodies for 20 minutes at 37° C. 1×105 isolated B cells from human PBMCs were added and incubated room temperature for an additional 20 minutes to allow for cell/antibody interaction. Stimulant (anti-human IgM+IL-4) or control (media alone+/−IL-4) was then added and cells were incubated for 72 hours at 37° C. The effect of antibodies on B cell IL-6 response was evaluated at 72 hours by ELISA and reported as % inhibition compared with no antibody control. Antibodies were evaluated at concentrations ranging 0.000013-0.0539 nM (0.002-8 ng/mL), serial dilution for IC50 evaluation of inhibition of B cell IL-6 response.

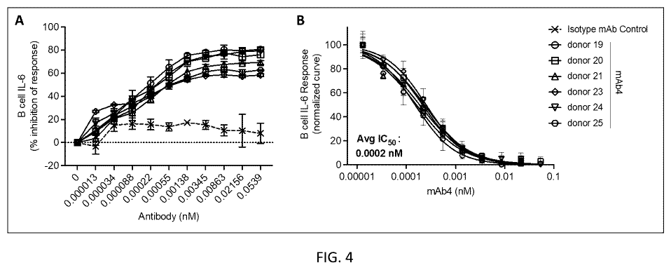

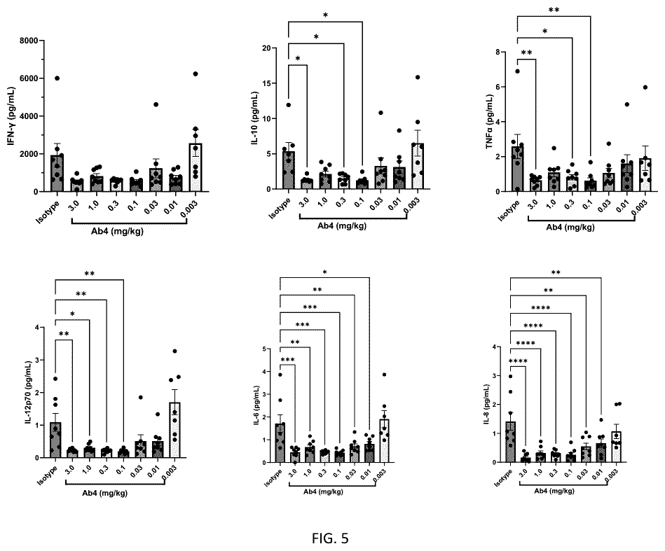

As shown in FIG. 4, mAb4 inhibited B cell IL-6 response to BCR stimulation, with inhibition ranging 60-80% (calculated as % IL-6 response with antibody vs. no antibody) and IC50 value 0.0002 nM (0.03 ng/mL), whereas hIgG4SP isotype control antibody had no impact on IL-6 response.

### Primary Human T Cells:

The effects of anti-human LAIR1 antibodies on primary human T cell cytokine response were evaluated. Primary human T cells were TCR-stimulated with anti-human CD3 and anti-human CD28 antibodies in the presence of anti-human LAIR1 antibodies at concentrations ranging 1 ng/mL-1 μg/mL. Specifically, T cells were incubated overnight with plate-bound anti-human CD3 antibody at 1 μg/mL and anti-human CD28 antibody at 311 g/mL at 37° C. CHO-K1 cells were seeded overnight in 96-well flat bottom tissue-culture sterile plates. When confluency reached 85-95%, cells were washed with RPMI/5% human serum and incubated with anti-human LAIR1 antibodies at indicated concentrations for 20 minutes at 37° C. Stimulated T cells were washed and resuspended in fresh RPMI/5% human serum. 1-1.5×105 T cells were then layered over CHO-K1 cells and incubated for 72 hours at 37° C. Following incubation, supernatants were harvested and evaluated for IFN-γ secretion via ELISA.

As shown in Table 14, all anti-human LAIR1 test antibodies inhibited IFN-γ response, with inhibition at top dose 1 μg/mL ranging 20-80% depending on donor, whereas isotype controls had no impact.

TABLE 14
In vitro agonistic activity of anti-human LAIR1 antibodies
on primary T cells following TCR stimulation.

| | Inhibition of IFN-γ response |
|---|---|
| | at 1 μg/mL |
| Antibody ID | (% inhibition vs. no antibody) |
| mAb1 | 35.67 |
| mAb3 | 31.23 |
| mAb2 | 45.8 |
| mAb4 | 41.13 |

### Example 5: In Vivo Activities of the Anti-Human LAIR1 Antibodies in Human PBMC Engrafted Mouse Model of Graft Versus Host Disease (GvHD)

To test the immune modulatory activity of the exemplified antibodies mAb1-mAb4, a humanized model of xenogeneic GvHD was utilized. Human immune cells recognize the mouse as foreign and mount an immune response resulting in significant increases in human pro-inflammatory cytokines, immune cell activation and expansion, and production of immunoglobulins. Importantly, the inflammatory response is driven by human cells and thus human specific treatments can be interrogated in this model.

Briefly, female NSG mice (NOD.Cg-Prkdcscid Il2rgtm1Wjl/SzJ, JAX Labs, Stock #05557) were housed 3 per cage at 72° C. under a 12 hour light:dark cycle and allowed food and water ad libitum. Human PBMCs were isolated from LRS tubes obtained from a single anonymous donor (San Diego Blood Bank) using SepMate 50 Ficoll preparation tubes according to the manufacturer's instructions (STEMCELL Technologies, Vancouver, BC). Freshly isolated PBMCs were suspended in Pedialyte solution at 1.2×108 cells/mL and mice were engrafted with 100 □L PBMCs suspension intravenously on day 0 (1.2×107/mouse, n=36). Mice were divided into 5 groups and dosed on days 1 and 8 with isotype control or mAbs 1-4 at 0.3 mg/kg subcutaneously (200 μL/mouse; n=7-8/group). On day 7, mice were briefly anesthetized with isoflurane and blood was obtained from the retro-orbital sinus. On day 14, mice were anesthetized again, blood collected by cardiac puncture, and mice were euthanized. Mice were weighed in a BSL2 hood and assessed for clinical signs of distress 2-3 times/week. Clinical signs common to this model are scruffy hair, hunched body, wasting, and labored breathing or movement. Blood from the 2 collections were clarified by centrifugation, and the resultant plasma was stored at −80° C. for future processing. Plasma cytokines were measured using the Human Pro-inflammatory 10-Vplex and IgM, IgA, and IgG using the Human Isotyping Panel (Meso Scale Discovery, Rockville, Maryland) according to the manufacturer's instructions.

Data were graphed and statistics were calculated using Prism Software (GraphPad, San Diego, CA). Differences in plasma analytes compared to isotype control were determined by 1-way ANOVA with Dunnett's post hoc test and considered significant if p<0.05.

In experiments performed essentially as described above, the exemplified anti-human LAIR1 antibodies significantly inhibited the pronounced increase in plasma human pro-inflammatory cytokines (IFN-γ, IL-10, and TNF-α) associated with disease progression in the GvHD model Additionally, the exemplified anti-human LAIR1 antibodies mAb1-mAb3 significantly reduced circulating immunoglobulins IgM and IgA, suggesting inhibitory effects on B-cells.

The results demonstrate the exemplified anti-human LAIR1 antibodies have immunomodulatory effects on human immune cells in a humanized mouse model of disease.

### Humanized Graft vs Host Disease:

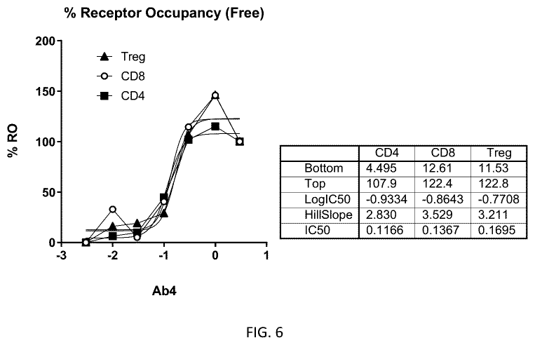

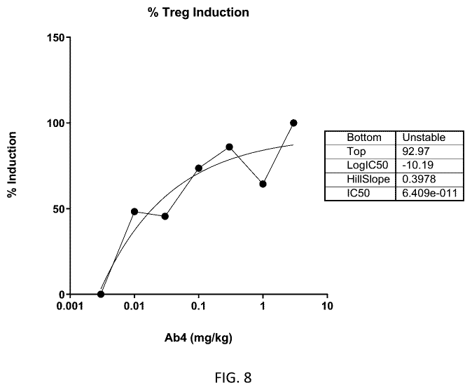

The antibody described herein as mAb4 was tested in NOD SCID Gamma2 chain−/− (NSG) humanized mice to assess its ability to inhibit human T cell function in an in vivo setting. Human peripheral blood mononuclear cells (PBMCs) were engrafted into NSG mice where the human immune cells recognize the mouse as foreign and mount an immune response resulting in Graft versus Host Disease (GvHD). The objective was to evaluate the ability of mAb4 to agonize LAIR1 and inhibit T cell activation, as measured by proinflammatory cytokine production and correlate these effects with drug exposure and immune cell receptor occupancy to assist with human dose projections. 1.2e7 human PBMCs were injected intravenously to NSG mice. mAb4 at half-log increments from 0.003-3 mg/kg or a human IgG4P isotype control (3 mg/kg) were dosed once subcutaneously twenty-four hours post cell engraftment and mice were euthanized on day 8. Blood was obtained on day 5 by retro-orbital sinus and day 8 by cardiac puncture and processed for analyses of serum cytokines (MSD human pro-inflammatory cytokine panel) and drug exposure (antigen capture ELISA). Spleens were harvested on day 8, processed to single cell splenocytes, and analyzed by FACS for immunophenotyping and receptor occupancy (RO). mAb4 dose dependently inhibited immune cell associated pro-inflammatory cytokines indicative of T-cell function inhibition (FIG. 5). These beneficial activities were supported mechanistically by post-mortem FACS analysis of splenocytes where dose dependent receptor occupancy of LAIR1 was observed on regulatory T cells (Tregs), CD4 and CD8 T cells (FIG. 6). Drug exposure measured on days 4 and 7 post dose demonstrated dose dependency similar to receptor occupancy. Interestingly, the PD responses were more potent than the exposure or RO would imply suggesting full RO is not required to elicit the beneficial effects of agonism. Additionally, an increase in the percentage of Tregs observed in the high dose groups offers another potential therapeutic mechanism by which LAIR1 agonism may prove advantageous in treating autoimmune diseases (FIG. 8).

### SUMMARY

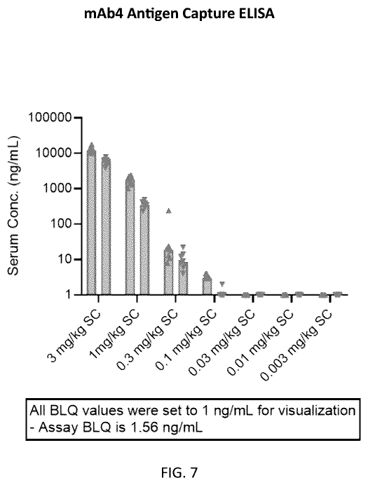

The pharmacodynamic activity of mAb4 was evaluated in a humanized mouse model of graft vs. host disease. In this model, mice lacking a complete immune system are engrafted with human donor immune cells. After engraftment the human immune cells mount an inflammatory attack on the mice. This is measured by production of human cytokines in the mouse peripheral blood. The LAIR1 agonist antibody, mAb4, was able to reduce the production of these cytokines in a dose-dependent manner (FIG. 5). This reduction was correlated with the amount of receptor occupancy (FIG. 6) and serum concentration of mAb4 (FIG. 7). Furthermore, mAb4 was able to increase the percent population of Treg cells in the spleen (FIG. 8), which could account for another mechanism of action in controlling the inflammatory response in this model.

### Example 6: In Vivo Studies of the Anti-Human LAIR1 Antibodies in Mouse Model of Spontaneous Lupus Nephritis

### Primary Pharmacodynamics

Type-I interferon Lupus Nephritis:

NZB/W F1 mice are used as a classical model of spontaneous lupus nephritis. To accelerate and synchronize disease induction, we injected the mice with an adeno-associated virus (AAV) that expressed mouse IFNα5. Therapies targeted against T and B cells have been shown to reduce disease severity in this model. The purpose of this study was to demonstrate whether a surrogate LAIR1 agonistic antibody can affect disease severity in a preclinical model of lupus nephritis.

### Lupus Model:

Female NZB/W F1 mice (Jackson Laboratories) were 10 weeks old upon arrival. All mice were housed 5 per cage and allowed to acclimate for 1 week prior to start of study. The mice were fed Teklad Irradiated Global 18% Protein Rodent Diet 2908 (Innotiv) and given water ad libitum. The mice were housed in 12-hour light/dark cycle with ambient temperature range at 68-79° F. Mice were sorted based on body weight and one day later (Day 0) the mice were injected intravenously with LacZ-AAV (non-diseased control, 1011 genome copies (GC)) or mouse IFNα5-AAV (3×1012 GC) in 100 μl PBS. The assigned treatment groups were: (1) LacZ-AAV induced, treated with PBS (s.c., BID starting on Day 7, n=5), (2) IFN-AAV induced, treated with IgG isotype, used as a surrogate antibody, (10 mg/kg s.c., BID starting on Day 7, n=10), (3) IFN-AAV induced, treated with surrogate antibody (10 mg/kg s.c. BID starting on Day 7, n=10), (4) IFN-AAV induced, treated with surrogate antibody (10 mg/kg s.c. BID starting on Day 21, n=10), and (5) IFN-AAV induced, treated with cyclophosphamide (15 mg/kg i.p. Q10D starting on Day 7, n=10). Serum and urine samples were collected at baseline and every 2 weeks during the study. Forty-two days after AAV injection, the mice were euthanized and body weights were collected. Both kidneys were collected and weighed as a pair. The right kidney was fixed in 10% neutral buffered formalin for 24-48 hours and then transferred to 70% alcohol.

### Albumin and Creatinine Assay:

To monitor renal function, micro-albumin concentration in urine (dilution 1:500-1:50,000) samples were determined by ELISA (Mouse Microalbumin ELISA kit, Kamiya Biomedical Co, Seattle, WA) according to the manufacturer's instructions. Urine creatinine was measured by using CREP2 enzymatic creatinine assay with a Cobas C501 clinical chemistry analyzer (Roche Diagnostics, USA) according to the manufacturer's instructions.

### Histology:

Kidneys from each mouse were embedded in paraffin, sectioned, and stained with hematoxylin & eosin PAS.

Histology scoring: Scoring of inflammation, glomerular changes, and tubular protein was based on the following criteria, when added together resulted in a total score.

Inflammation: Scores of 0-3 were based on a combination of number of areas affected and amount of area affected.

Glomerular scores: Glomerular scoring (0-6) was based on assessment of the glomeruli in the outer one-half of the cortex, and on the most frequent grade encountered in this region as there was variability between glomeruli within each kidney:—
Grade 1—Minimally increased cellularity and/or mesangial expansion+/−minimal increase in glomerular size (less than 2-fold).
Grade 2—Mildly increased cellularity and mesangial expansion with most glomeruli at least 2-fold greater in size.
Grade 3—Moderately increased cellularity and some areas of prominent mesangial expansion and/or capillary proliferation in most affected glomeruli with up to 3-fold increase in glomerular size.
Grade 4—Markedly increased cellularity and some areas of prominent mesangial expansion and/or capillary proliferation in most affected glomeruli with up to 4-fold increase in glomerular size; rare sclerotic glomeruli; may have hypertrophy of parietal cells.
Grade 5—Above with <25% of glomeruli sclerotic and/or capillary proliferation in most affected glomeruli; up to 5-fold increase in glomerular size.
Grade 6—Above with >25% of glomeruli sclerotic, characterized in part by decreased tuft cellularity+/−periglomerular fibrosis+/−hypertrophy of parietal cells.

PAS scores: PAS scores of 0-3 were based upon the presence of increased staining of the glomerular mesangial matrix in the outer one-half of the cortex, compared to control sections cut at the same thickness.
Grade 1—minimally increased mesangial staining of scattered glomeruli.
Grade 2—more extensive expansion (and therefore PAS staining) of the mesangium affecting more of the glomeruli.

Grade 3—pronounced expansion of the mesangium in most of the glomeruli.

Tubular protein scores: Scores of 0-3 were based on the percentage of tubules containing proteinaceous fluid.

Grade 1—<25% of tubules affected.

Grade 2-25-50% of tubules affected.

Grade 3—>50% of tubules affected.

Total histology scores were calculated using the sum of the scores for the 4 parameters.

### Statistical Analysis:

Statistical analyses were conducted using One-Way ANOVA followed by Dunnett's post-test comparison vs. IFNα-induced treated with IgG isotype.

### SUMMARY

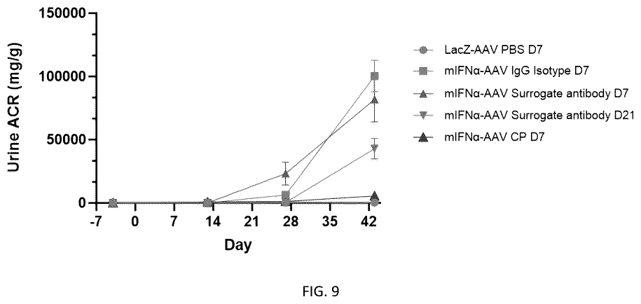

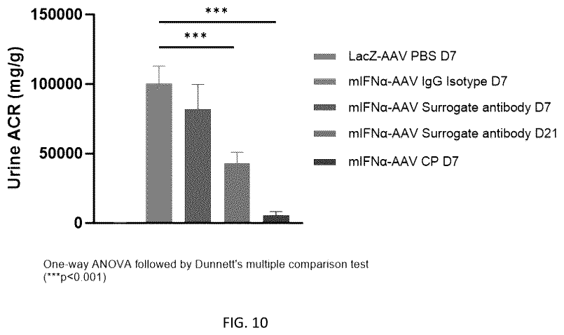

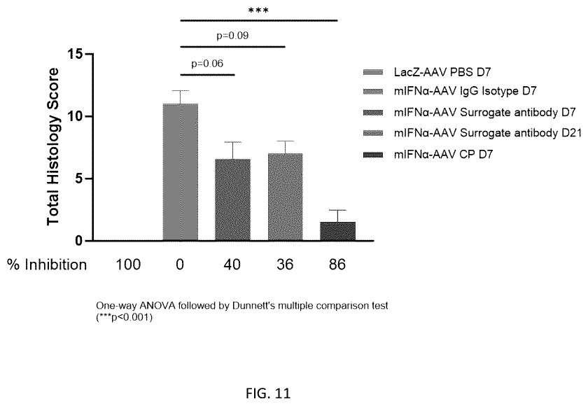

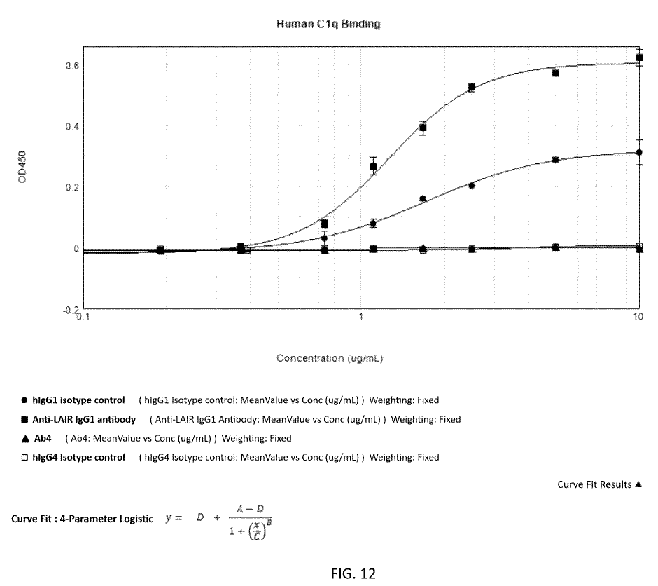

Administration of mIFNα-AAV to NZB/W F1 mice induced lupus nephritis characterized by increases in urine ACR levels and histology scores in the IgG isotype group as compared to mice administered non-pathogenic LacZ-AAV (FIGS. 5 and 7). Compared to the IgG isotype group, therapeutic treatment with surrogate antibody starting on day 21 (D21) reduced urine ACR levels by end of study on day 44, but was not as effective as the positive control, cyclophosphamide (CP) administered starting on day 7 (D7) (FIGS. 9 and 10). However, preventative treatment with surrogate antibody starting on D7 did not significantly reduce urine ACR (FIGS. 9 and 10). Histological evaluation of kidneys from the mice demonstrated that surrogate antibody reduced histology scores by 40% and 36% when treatment was initiated on D7 and D21, respectively (FIG. 11). The effects were not statistically significant with p values of 0.06 and 0.09, respectively, compared to IgG isotype control (FIG. 11).

The results demonstrate that a surrogate agonistic antibody can modulate disease in a pre-clinical model of lupus nephritis.

### Example 7: Epitope Mapping of Parental mAb4 to the LAIR1 Extracellular Domain (ECD) Protein by Hydrogen Deuterium Exchange Mass Spectrometry

Hydrogen deuterium exchange coupled with mass spectrometry (HDX-MS) was performed to determine where parental mAb4 binds the ECD of LAIR1.

Peptide identification for LAIR1 ECD was performed on a Waters Synapt G2Si (Waters Corporation) instrument using 3.5 μg of LAIR1 ECD protein at zero exchange (1:10 dilution in 0.1× phosphate buffered saline in H₂O) using nepenthesin II (Nep II) for digestion. The mass spectrometer was set in HDMSe (Mobility ESI+ mode) using a mass acquisition range of m/z 255.00-1950.00 with a scan time of 0.4 s. Data was processed using PLGS 2.3.03 (Waters Corporation). For the exchange experiments, the complex of LAIR1 ECD protein with mAb4 was prepared at the molar ratio of 1:1.2 in 10 mM sodium phosphate buffer, pH 7.4 containing 150 mM NaCl (1×PBS buffer). The experiment was initiated by adding 25 μL of D20 buffer containing 0.1×PBS to 2.5 μl of LAIR1 ECD (0.7 mg/mL) or LAIR1 ECD+mAb4 complex at 15° C. for various amounts of time (0s, 10s, 2 min, 10 min and 60 min) using a custom TECAN sample preparation system (Espada et al. 2019). The reaction was quenched using equal volume of was 0.32M TCEP, 0.1M phosphate pH 2.5 for two minutes at 4° C. and immediately frozen at −70° C. The sample injection system was comprised of a UR3 robot, a LEAP PAL3 HDX autosampler, and a HPLC system interfaced with a Waters Synapt G2Si (Waters Corporation), with modification as described (Espada et al., 2019, https://pubmed.ncbi.nlm.nih.gov/31724102/). The LC mobile phases consisted of water (A) and acetonitrile (B), each containing 0.2% formic acid. Each sample was thawed using 50 μL of 0.2% formic acid in water, pH 2.5, for 1 min and injected on to a Nep II column for digestion at 4° C. with mobile phase A at a flow rate of 250 μL/min for 2.5 minutes. The resulting peptides were trapped on a Waters BEH Vanguard Pre-column at 4° C., and chromatographically separated using a Waters Acquity UPLC BEH C18 analytical column at 4° C. with a flow rate of 200 μL/min and a gradient of 3%-85% mobile phase B over 7 minutes and directed into mass spectrometer for mass analysis. The Synapt G2Si was calibrated with Glu-fibrinopeptide (Waters Corporation) prior to use. Mass spectra were acquired over the m/z range of 255 to 1950 in HDMS mode, with the lock mass m/z of 556.2771 (Leucine Enkephalin, Waters Corporation). The relative deuterium incorporation for each peptide was determined by processing the MS data for deuterated samples along with the undeuterated control using the identified peptide list in DynamX 3.0 (Waters Corporation). Peptides from the free and bound states of RBD were compared for deuterium incorporation differences to identify protected regions indicative of the binding epitope.

Sequence coverage across the LAIR1 ECD was 77%. For parental mAb4, decrease in deuterium uptake upon binding to LAIR1 ECD was observed in residues 26-41 (FVCRGPVGVQTFRLER) (SEQ ID NO: 32) and 53-68 (VSQASPSESEARFRI) (SEQ ID NO:33) pointing to the probable epitope regions. Parental mAb4 sequences are shown in Table 1 above.

### Example 6: C1q Binding Results

A 96-well microplate was coated with 100 μL/well of each antibody diluted in DPBS (Dulbecco's HyClone) with a concentration range of 10 μg/mL to 0.19 μg/mL. Testing was performed in duplicate wells. The plate was sealed and incubated overnight at 4° C. The coating reagent was removed from each well, and 200 μL/well of casein blocking reagent (Thermo) was added. The plate was sealed and incubated for 2 hours at room temperature (RT). Each well was washed 3 times with wash buffer (1×TBE with 0.05% Tween 20). One hundred microliters per well of Human C1q (MS Biomedical) at 10 μg/mL diluted in casein blocking reagent was added and incubated for 3 hours at RT. The plate was then washed three times with wash buffer before 100 μL/well of a 1:800 times dilution of Sheep anti-human C1q-HRP (Abcam #ab46191) in casein blocker was added and incubated for 1 hour at RT. The plate was washed 6 times with wash buffer, and 100 μL/well of TMB Substrate (Pierce) was added to each well and incubated for 7 minutes. One hundred microliters of 1 N HCl was added to each well to stop the reaction. Optical density was immediately measured using a colorimetric microplate reader set to 450 nm. The result shows that mAb4 and the humanized IgG4-P isotype control antibody, and human IgG1 isotype control antibody had no binding to the complement component C1q. The anti-LAIR1 IgG1 antibody, and human IgG1 isotype control antibody, did bind complement component C1q, as expected.

The results indicated that mAb4 is unlikely to elicit Fc-mediated effector function response in vivo.

SEQUENCE LISTING

| SEQ | |
|---|---|
| ID | |
| NO | Sequence |
| 1 | TAFGGSISGHYWS |
| 2 | RIYPSGGTN |
| 3 | VRDYWDDYYGMDV |
| 4 | RSSQSLLHSDGFNYLD |
| 5 | YLGSNRAS |
| 6 | MQAYQTPRT |
| 7 | QVQLQESGPGLVKPSETLSLTCTAFGGSISGHYWSWIRQPAGKGLE |
| | WIGRIYPSGGTNKNPSLKSRVTMSVDTSKNQFSLKLSSVTAADTAV |
| | YYCVRDYWDDYYGMDVWGQGTTVTVSS |
| 8 | DIVMTQSPLSLPVTPGEPASISCRSSQSLLHSDGFNYLDWYLQKPG |
| | QSPQLLIYLGSNRASGVPDRFSGSGSGTDFTLKISRVEAEDVGVYY |
| | CMQAYQTPRTFGQGTKVEIK |
| 9 | QVQLQESGPGLVKPSETLSLTCTAFGGSISGHYWSWIRQPAGKGLE |
| | WIGRIYPSGGTNKNPSLKSRVTMSVDTSKNQFSLKLSSVTAADTAV |
| | YYCVRDYWDDYYGMDVWGQGTTVTVSSASTKGPSVFPLAPSSRS |
| | TSESTAALGCLVKDYFPEPVTVSWNSGALTSGVHTFPAVLQSSGLY |
| | SLSSVVTVPSSNFGTQTYTCNVDHKPSNTKVDKTVERKCCVECPPC |
| | PAPPVAGPSVFLFPPKPKDTLMISRTPEVTCVVVDVSHEDPEVQFN |
| | WYVDGMEVHNAKTKPREEQFNSTFRVVSVLTVVHQDWLNGKEY |
| | KCKVSNKGLPAPIEKTISKTKGQPREPQVYTLPPSREEMTKNQVSL |
| | TCLVKGFYPSDIAVEWESNGQPENNYKTTPPMLDSDGSFFLYSKLT |
| | VDKSRWQQGNVFSCSVMHEALHNHYTQKSLSLSPG |
| 10 | DIVMTQSPLSLPVTPGEPASISCRSSQSLLHSDGFNYLDWYLQKPG |
| | QSPQLLIYLGSNRASGVPDRFSGSGSGTDFTLKISRVEAEDVGVYY |
| | CMQAYQTPRTFGQGTKVEIKRTVAAPSVFIFPPSDEQLKSGTASVV |
| | CLLNNFYPREAKVQWKVDNALQSGNSQESVTEQDSKDSTYSLSST |
| | LTLSKADYEKHKVYACEVTHQGLSSPVTKSFNRGEC |
| 11 | CAGGTGCAGCTGCAGGAGTCGGGCCCAGGACTGGTGAAGCCTT |
| | CGGAGACCCTGTCCCTCACCTGTACTGCCTTTGGTGGCTCCATC |
| | AGTGGTCACTACTGGAGCTGGATCCGGCAGCCCGCCGGGAAGG |
| | GACTGGAGTGGATTGGGCGTATTTATCCTAGTGGGGGCACCAA |
| | CAAAAACCCCTCCCTCAAGAGTCGCGTCACCATGTCAGTAGAC |
| | ACGTCCAAGAATCAGTTCTCCCTGAAGCTGAGTTCTGTGACCGC |
| | CGCGGACACGGCCGTGTATTATTGTGTGAGAGATTACTGGGAC |
| | GACTACTATGGTATGGACGTCTGGGGCCAAGGGACCACGGTCA |
| | CCGTCTCCTCAGCCTCCACCAAGGGCCCATCGGTCTTCCCCCTG |
| | GCGCCCAGCTCCAGGAGCACCTCCGAGAGCACAGCCGCCCTGG |
| | GCTGCCTGGTCAAGGACTACTTCCCCGAACCGGTGACGGTGTCG |
| | TGGAACTCAGGCGCCCTGACCAGCGGCGTGCACACCTTCCCGG |
| | CTGTCCTACAGTCCTCAGGACTCTACTCCCTCAGCAGCGTGGTG |
| | ACCGTGCCCTCCAGCAACTTCGGCACCCAGACCTACACCTGCAA |
| | CGTAGATCACAAGCCCAGCAACACCAAGGTGGACAAGACAGTT |
| | GAGCGCAAATGTTGTGTCGAGTGCCCACCGTGCCCAGCACCAC |
| | CTGTGGCAGGACCGTCAGTCTTCCTCTTCCCCCCAAAACCCAAG |
| | GACACCCTCATGATCTCCCGGACCCCTGAGGTCACGTGCGTGGT |
| | GGTGGACGTGAGCCACGAAGACCCCGAGGTCCAGTTCAACTGG |
| | TACGTGGACGGCATGGAGGTGCATAATGCCAAGACAAAGCCAC |
| | GGGAGGAGCAGTTCAACAGCACGTTCCGTGTGGTCAGCGTCCT |
| | CACCGTCGTGCACCAGGACTGGCTGAACGGCAAGGAGTACAAG |
| | TGCAAGGTCTCCAACAAAGGCCTCCCAGCCCCCATCGAGAAAA |
| | CCATCTCCAAAACCAAAGGGCAGCCCCGAGAACCACAGGTGTA |
| | CACCCTGCCCCCATCCCGGGAGGAGATGACCAAGAACCAGGTC |
| | AGCCTGACCTGCCTGGTCAAAGGCTTCTACCCCAGCGACATCGC |
| | CGTGGAGTGGGAGAGCAATGGGCAGCCGGAGAACAACTACAA |
| | GACCACACCTCCCATGCTGGACTCCGACGGCTCCTTCTTCCTCT |
| | ACAGCAAGCTCACCGTGGACAAGAGCAGGTGGCAGCAGGGGA |
| | ACGTCTTCTCATGCTCCGTGATGCATGAGGCTCTGCACAACCAC |
| | TACACACAGAAGAGCCTCTCCCTGTCTCCGGGTTGA |
| 12 | GATATTGTGATGACTCAGTCTCCACTCTCCCTGCCCGTCACCCCT |
| | GGAGAGCCGGCCTCCATCTCCTGCAGGTCTAGTCAGAGCCTCCT |
| | GCATAGTGATGGATTCAACTATTTGGATTGGTACCTGCAGAAGC |
| | CAGGGCAGTCTCCACAGCTCCTGATCTATCTGGGTTCTAATCGG |
| | GCCTCCGGGGTCCCTGACAGGTTCAGTGGCAGTGGATCAGGCA |
| | CAGATTTTACACTGAAAATCAGCAGAGTGGAGGCTGAGGATGT |
| | TGGGGTTTATTACTGCATGCAAGCTTATCAAACTCCTCGGACGT |
| | TCGGCCAAGGGACCAAGGTGGAAATCAAACGAACCGTGGCTGC |
| | ACCATCTGTCTTCATCTTCCCGCCATCTGATGAGCAGTTGAAAT |
| | CTGGAACTGCCTCTGTTGTGTGCCTGCTGAATAACTTCTATCCC |
| | AGAGAGGCCAAAGTACAGTGGAAGGTGGATAACGCCCTCCAAT |
| | CGGGTAACTCCCAGGAGAGTGTCACAGAGCAGGACAGCAAGGA |
| | CAGCACCTACAGCCTCAGCAGCACCCTGACGCTGAGCAAAGCA |
| | GACTACGAGAAACACAAAGTCTACGCCTGCGAAGTCACCCATC |
| | AGGGCCTGAGCTCGCCCGTCACAAAGAGCTTCAACAGGGGAGA |
| | GTGC |
| 13 | TVSGGSISNNYWS |
| 14 | RMYSSGSTQ |
| 15 | GPNWGSPYYGVDV |
| 16 | RSSPSLLHSDGYNWLD |
| 17 | QEEDLPRPSISAEPGTVIPLGSHVTFVCRGPVGVQTFRLERESRST |
| | YNDTEDVSQASPSESEARFRIDSVSEGNAGPYRCIYYKPPKWSEQS |
| | DYLELLVKETSGGPDSPDTEPGSSAGPTQRPSDNSHNEHAPASQGL |
| | KAEHENLYFQ |
| 18 | MQAHQTPFT |
| 19 | QVQLQESGPGLVKPSETLSLTCTVSGGSISNNYWSWIRQPAGKGLE |
| | WIGRMYSSGSTQKNPSLKSRVTMSVDTSKNQFSLKLSSVTAADTA |
| | VYYCVRGPNWGSPYYGVDVWGQGTTVTVSS |
| 20 | DIVMTQSPLSLPVTPGEPASISCRSSPSLLHSDGYNWLDWYLQKPG |
| | QSPQLLIYLGSNRASGVPDRFSGSGSGTDFTLKISRVEAEDVGVYY |
| | CMQAHQTPFTFGQGTKVEIK |
| 21 | QVQLQESGPGLVKPSETLSLTCTVSGGSISNNYWSWIRQPAGKGLE |
| | WIGRMYSSGSTQKNPSLKSRVTMSVDTSKNQFSLKLSSVTAADTA |
| | VYYCVRGPNWGSPYYGVDVWGQGTTVTVSSASTKGPSVFPLAPS |
| | SRSTSESTAALGCLVKDYFPEPVTVSWNSGALTSGVHTFPAVLQSS |
| | GLYSLSSVVTVPSSNFGTQTYTCNVDHKPSNTKVDKTVERKCCVE |
| | CPPCPAPPVAGPSVFLFPPKPKDTLMISRTPEVTCVVVDVSHEDPE |
| | VQFNWYVDGMEVHNAKTKPREEQFNSTFRVVSVLTVVHQDWLNG |
| | KEYKCKVSNKGLPAPIEKTISKTKGQPREPQVYTLPPSREEMTKNQ |
| | VSLTCLVKGFYPSDIA VEWESNGQPENNYKTTPPMLDSDGSFFLY |
| | SKLTVDKSRWQQGNVFSCSVMHEALHNHYTQKSLSLSPG |
| 22 | DIVMTQSPLSLPVTPGEPASISCRSSPSLLHSDGYNWLDWYLQKPG |
| | QSPQLLIYLGSNRASGVPDRFSGSGSGTDFTLKISRVEAEDVGVYY |
| | CMQAHQTPFTFGQGTKVEIKRTVAAPSVFIFPPSDEQLKSGTASVV |
| | CLLNNFYPREAKVQWKVDNALQSGNSQESVTEQDSKDSTYSLSST |
| | LTLSKADYEKHKVYACEVTHQGLSSPVTKSFNRGEC |
| 23 | CAGGTGCAGCTGCAGGAGTCGGGCCCAGGACTGGTGAAGCCTT |
| | CGGAGACCCTGTCCCTCACCTGCACTGTCTCTGGTGGCTCCATC |
| | AGTAATAACTACTGGAGTTGGATCCGGCAGCCCGCCGGTAAGG |
| | GGCTGGAGTGGATTGGGCGTATGTATTCCAGTGGGAGCACCCA |
| | GAAAAACCCCTCCCTCAAGAGTCGAGTCACCATGTCAGTAGAC |
| | ACGTCCAAAAACCAGTTCTCCCTGAAGCTGTCCTCTGTGACCGC |
| | CGCGGACACGGCCGTGTATTATTGTGTGAGAGGCCCTAACTGG |
| | GGATCCCCCTACTACGGTGTGGACGTCTGGGGCCAAGGGACCA |
| | CGGTCACCGTCTCCTCAGCCTCCACCAAGGGCCCATCGGTCTTC |
| | CCCCTGGCGCCCAGCTCCAGGAGCACCTCCGAGAGCACAGCCG |
| | CCCTGGGCTGCCTGGTCAAGGACTACTTCCCCGAACCGGTGACG |
| | GTGTCGTGGAACTCAGGCGCCCTGACCAGCGGCGTGCACACCTT |
| | CCCGGCTGTCCTACAGTCCTCAGGACTCTACTCCCTCAGCAGCG |
| | TGGTGACCGTGCCCTCCAGCAACTTCGGCACCCAGACCTACACC |
| | TGCAACGTAGATCACAAGCCCAGCAACACCAAGGTGGACAAGA |
| | CAGTTGAGCGCAAATGTTGTGTCGAGTGCCCACCGTGCCCAGCA |
| | CCACCTGTGGCAGGACCGTCAGTCTTCCTCTTCCCCCCAAAACC |
| | CAAGGACACCCTCATGATCTCCCGGACCCCTGAGGTCACGTGCG |
| | TGGTGGTGGACGTGAGCCACGAAGACCCCGAGGTCCAGTTCAA |
| | CTGGTACGTGGACGGCATGGAGGTGCATAATGCCAAGACAAAG |
| | CCACGGGAGGAGCAGTTCAACAGCACGTTCCGTGTGGTCAGCG |
| | TCCTCACCGTCGTGCACCAGGACTGGCTGAACGGCAAGGAGTA |
| | CAAGTGCAAGGTCTCCAACAAAGGCCTCCCAGCCCCCATCGAG |
| | AAAACCATCTCCAAAACCAAAGGGCAGCCCCGAGAACCACAGG |
| | TGTACACCCTGCCCCCATCCCGGGAGGAGATGACCAAGAACCA |
| | GGTCAGCCTGACCTGCCTGGTCAAAGGCTTCTACCCCAGCGACA |
| | TCGCCGTGGAGTGGGAGAGCAATGGGCAGCCGGAGAACAACTA |
| | CAAGACCACACCTCCCATGCTGGACTCCGACGGCTCCTTCTTCC |
| | TCTACAGCAAGCTCACCGTGGACAAGAGCAGGTGGCAGCAGGG |
| | GAACGTCTTCTCATGCTCCGTGATGCATGAGGCTCTGCACAACC |
| | ACTACACACAGAAGAGCCTCTCCCTGTCTCCGGGTTGA |
| 24 | GATATTGTGATGACTCAGTCTCCACTCTCCCTGCCCGTCACCCCT |
| | GGAGAGCCGGCCTCCATCTCCTGCAGGTCTAGTCCGAGCCTCCT |
| | GCATAGTGATGGATACAACTGGTTGGATTGGTACCTGCAGAAG |
| | CCAGGGCAGTCTCCACAGCTCCTGATCTATTTGGGTTCTAATCG |
| | GGCCTCCGGGGTCCCTGACAGGTTCAGTGGCAGTGGATCAGGC |
| | ACAGATTTTACACTGAAAATCAGCAGAGTGGAGGCTGAGGATG |
| | TTGGGGTTTATTACTGCATGCAAGCTCACCAAACTCCATTCACT |
| | TTCGGCCAGGGGACCAAGGTGGAAATCAAACGAACCGTGGCTG |
| | CACCATCTGTCTTCATCTTCCCGCCATCTGATGAGCAGTTGAAA |
| | TCTGGAACTGCCTCTGTTGTGTGCCTGCTGAATAACTTCTATCCC |
| | AGAGAGGCCAAAGTACAGTGGAAGGTGGATAACGCCCTCCAAT |
| | CGGGTAACTCCCAGGAGAGTGTCACAGAGCAGGACAGCAAGGA |
| | CAGCACCTACAGCCTCAGCAGCACCCTGACGCTGAGCAAAGCA |
| | GACTACGAGAAACACAAAGTCTACGCCTGCGAAGTCACCCATC |
| | AGGGCCTGAGCTCGCCCGTCACAAAGAGCTTCAACAGGGGAGA |
| | GTGC |
| 25 | QVQLQESGPGLVKPSETLSLTCTAFGGSISGHYWSWIRQPAGKGLE |
| | WIGRIYPSGGTNKNPSLKSRVTMSVDTSKNQFSLKLSSVTAADTAV |
| | YYCVRDYWDDYYGMDVWGQGTTVTVSSASTKGPSVFPLAPCSRS |
| | TSESTAALGCLVKDYFPEPVTVSWNSGALTSGVHTFPAVLQSSGLY |
| | SLSSVVTVPSSSLGTKTYTCNVDHKPSNTKVDKRVESKYGPPCPPC |
| | PAPEFLGGPSVFLFPPKPKDTLMISRTPEVTCVVVDVSQEDPEVQF |
| | NWYVDGVEVHNAKTKPREEQFNSTYRVVSVLTVLHQDWLNGKEY |
| | KCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLT |
| | VDKSRWQEGNVFSCSVMHEALHNHYTQKSLSLSLG |
| 26 | CAGGTGCAGCTGCAGGAGTCGGGCCCAGGACTGGTGAAGCCTT |
| | CGGAGACCCTGTCCCTCACCTGTACTGCCTTTGGTGGCTCCATC |
| | AGTGGTCACTACTGGAGCTGGATCCGGCAGCCCGCCGGGAAGG |
| | GACTGGAGTGGATTGGGCGTATTTATCCTAGTGGGGGCACCAA |
| | CAAAAACCCCTCCCTCAAGAGTCGCGTCACCATGTCAGTAGAC |
| | ACGTCCAAGAATCAGTTCTCCCTGAAGCTGAGTTCTGTGACCGC |
| | CGCGGACACGGCCGTGTATTATTGTGTGAGAGATTACTGGGAC |
| | GACTACTATGGTATGGACGTCTGGGGCCAAGGGACCACGGTCA |
| | CCGTCTCCTCAGCTAGCACCAAGGGCCCATCGGTCTTCCCACTA |
| | GCGCCCTGCTCCAGGAGCACCTCCGAGAGCACAGCCGCCCTGG |
| | GCTGCCTGGTCAAGGACTACTTCCCCGAACCGGTGACGGTGTCG |
| | TGGAACTCAGGCGCCCTGACCAGCGGCGTGCACACCTTCCCGG |
| | CTGTCCTACAGTCCTCAGGACTCTACTCCCTCAGCAGCGTGGTG |
| | ACCGTGCCCTCCAGCAGCTTGGGCACGAAGACCTACACCTGCA |
| | ACGTAGATCACAAGCCCAGCAACACCAAGGTGGACAAGAGAGT |
| | TGAGTCCAAATATGGTCCCCCATGCCCACCCTGCCCAGCACCTG |
| | AGTTTCTGGGGGGACCATCAGTCTTCCTGTTCCCCCCAAAACCC |
| | AAGGACACTCTCATGATCTCCCGGACCCCTGAGGTCACGTGCGT |
| | GGTGGTGGACGTGAGCCAGGAAGACCCCGAGGTCCAGTTCAAC |
| | TGGTACGTGGATGGCGTGGAGGTGCATAATGCCAAGACAAAGC |
| | CGCGGGAGGAGCAGTTCAACAGCACGTACCGTGTGGTCAGCGT |
| | CCTCACCGTCCTGCACCAGGACTGGCTGAACGGCAAGGAGTAC |
| | AAGTGCAAGGTCTCCAACAAAGGCCTCCCGTCCTCCATCGAGA |
| | AAACCATCTCCAAAGCCAAAGGGCAGCCCCGAGAGCCACAGGT |
| | GTACACCCTGCCCCCATCCCAGGAGGAGATGACCAAGAACCAG |
| | GTCAGCCTGACCTGCCTGGTCAAAGGCTTCTACCCCAGCGACAT |
| | CGCCGTGGAGTGGGAAAGCAATGGGCAGCCGGAGAACAACTAC |
| | AAGACCACGCCTCCCGTGCTGGACTCCGACGGCTCCTTCTTCCT |
| | CTACAGCAGGCTAACCGTGGACAAGAGCAGGTGGCAGGAGGG |
| | GAATGTCTTCTCATGCTCCGTGATGCATGAGGCTCTGCACAACC |
| | ACTACACACAGAAGAGCCTCTCCCTGTCTCTGGGT |
| 27 | QVQLQESGPGLVKPSETLSLTCTVSGGSISNNYWSWIRQPAGKGLE |
| | WIGRMYSSGSTQKNPSLKSRVTMSVDTSKNQFSLKLSSVTAADTA |
| | VYYCVRGPNWGSPYYGVDVWGQGTTVTVSSASTKGPSVFPLAPC |
| | SRSTSESTAALGCLVKDYFPEPVTVSWNSGALTSGVHTFPAVLQSS |
| | GLYSLSSVVTVPSSSLGTKTYTCNVDHKPSNTKVDKRVESKYGPPC |
| | PPCPAPEFLGGPSVFLFPPKPKDTLMISRTPEVTCVVVDVSQEDPE |
| | VQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYS |
| | RLTVDKSRWQEGNVFSCSVMHEALHNHYTQKSLSLSLG |
| 28 | CAGGTGCAGCTGCAGGAGTCGGGCCCAGGACTGGTGAAGCCTT |
| | CGGAGACCCTGTCCCTCACCTGCACTGTCTCTGGTGGCTCCATC |
| | AGTAATAACTACTGGAGTTGGATCCGGCAGCCCGCCGGTAAGG |
| | GGCTGGAGTGGATTGGGCGTATGTATTCCAGTGGGAGCACCCA |
| | GAAAAACCCCTCCCTCAAGAGTCGAGTCACCATGTCAGTAGAC |
| | ACGTCCAAAAACCAGTTCTCCCTGAAGCTGTCCTCTGTGACCGC |
| | CGCGGACACGGCCGTGTATTATTGTGTGAGAGGCCCTAACTGG |
| | GGATCCCCCTACTACGGTGTGGACGTCTGGGGCCAAGGGACCA |
| | CGGTCACCGTCTCCTCAGCTAGCACCAAGGGCCCATCGGTCTTC |
| | CCACTAGCGCCCTGCTCCAGGAGCACCTCCGAGAGCACAGCCG |
| | CCCTGGGCTGCCTGGTCAAGGACTACTTCCCCGAACCGGTGACG |
| | GTGTCGTGGAACTCAGGCGCCCTGACCAGCGGCGTGCACACCTT |
| | CCCGGCTGTCCTACAGTCCTCAGGACTCTACTCCCTCAGCAGCG |
| | TGGTGACCGTGCCCTCCAGCAGCTTGGGCACGAAGACCTACAC |
| | CTGCAACGTAGATCACAAGCCCAGCAACACCAAGGTGGACAAG |
| | AGAGTTGAGTCCAAATATGGTCCCCCATGCCCACCCTGCCCAGC |
| | ACCTGAGTTTCTGGGGGGACCATCAGTCTTCCTGTTCCCCCCAA |
| | AACCCAAGGACACTCTCATGATCTCCCGGACCCCTGAGGTCACG |
| | TGCGTGGTGGTGGACGTGAGCCAGGAAGACCCCGAGGTCCAGT |
| | TCAACTGGTACGTGGATGGCGTGGAGGTGCATAATGCCAAGAC |
| | AAAGCCGCGGGAGGAGCAGTTCAACAGCACGTACCGTGTGGTC |
| | AGCGTCCTCACCGTCCTGCACCAGGACTGGCTGAACGGCAAGG |
| | AGTACAAGTGCAAGGTCTCCAACAAAGGCCTCCCGTCCTCCATC |
| | GAGAAAACCATCTCCAAAGCCAAAGGGCAGCCCCGAGAGCCAC |
| | AGGTGTACACCCTGCCCCCATCCCAGGAGGAGATGACCAAGAA |
| | CCAGGTCAGCCTGACCTGCCTGGTCAAAGGCTTCTACCCCAGCG |
| | ACATCGCCGTGGAGTGGGAAAGCAATGGGCAGCCGGAGAACA |
| | ACTACAAGACCACGCCTCCCGTGCTGGACTCCGACGGCTCCTTC |
| | TTCCTCTACAGCAGGCTAACCGTGGACAAGAGCAGGTGGCAGG |
| | AGGGGAATGTCTTCTCATGCTCCGTGATGCATGAGGCTCTGCAC |
| | AACCACTACACACAGAAGAGCCTCTCCCTGTCTCTGGGT |
| 29 | MSPHPTALLGLVLCLAQTIHTQEEDLPRPSISAEPGTVIPLGSHVT |
| | FVCRGPVGVQTFRLERDSRSTYNDTEDVSQASPSESEARFRIDSVR |
| | EGNAGLYRCIYYKPPKWSEQSDYLELLVKESSGGPDSPDTEPGSSA |
| | GPTQRPSDNSHNEHAPASQGLKAEHLYILIGVSVVFLFCLLLLVLF |
| | CLHRQNQIKQGPPRSKDEEQKPQQRPDLAVDVLERTADKATVNGLP |
| | EKDRETDTSALAAGSSQEVTYAQLDHWALTQRTARAVSPQSTKPMA |
| | ESITYAAVARH |
| 30 | QEEDLPRPSISAEPGTVIPLGSHVTFVCRGPVGVQTFRLERESRST |
| | YNDTEDVSQASPSESEARFRIDSVSEGNAGPYRCIYYKPPKWSEQS |
| | DYLELLVKETSGGPDSPDTEPGSSAGPTQRPSDNSHNEHAPASQGL |
| | KAEHENLYFQGEPKSSDKTHTCPPCPAPELLGGPSVFLFPPKPKDT |
| | LMISRTPEVTCVVVDVSHEDPEVKFNWYVDGVEVHNAKTKPREEQ |
| | YNSTYRVVSVLTVLHQDWLNGKEYKCKVSNKALPAPIEKTISKAK |
| | GQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEA |
| | LHNHYTQKSLSLSPGKHHHHHH |
| 31 | QEGALPRPSISAEPGTVISPGSHVTFMCRGPVGVQTFRLERED |
| | RAKYKDSYNVFRLGPSESEARFHIDSVSEGNAGLYRCLYYKP |
| | PGWSEHSDFLELLVKESSGGPDSPDTEPGSSAGTVPGTEASGF |
| | DAPENLYFQGEPKSSDKTHTCPPCPAPELLGGPSVFLFPPKPK |
| | DTLMISRTPEVTCVVVDVSHEDPEVKFNWYVDGVEVHNAK |
| | TKPREEQYNSTYRVVSVLTVLHQDWLNGKEYKCKVSNKAL |
| | PAPIEKTISKAKGQPREPQVYTLPPSRDELTKNQVSLTCLVKG |
| | FYPSDIAVEWESNGQPENNYKTTPPVLDSDGSFFLYSKLTVD |
| | KSRWQQGNVFSCSVMHEALHNHYTQKSLSLSPGK |
| 32 | FVCRGPVGVQTFRLER |
| 33 | VSQASPSESEARFRI |
| 34 | TVSGGSISNNYWS |
| 35 | RMYSSGSTN |
| 36 | ARGPNWGSPYYGVDV |

| | |
|---|---|
| 37 | RSSQSLLHSDGYNYLD |
| 38 | MQALQTPFT |
| 39 | QVQLQESGPGLVKPSETLSLTCTVSGGSISNNYWSWIRQSAGKGLE |
| | WIGRMYSSGSTNKNPSLKSRVTMSVDTSKNQFSLKLTSVTAADTA |
| | VYYCARGPNWGSPYYGVDVWGQGTTVTVSSASTKGPSVFPLAPS |
| | SKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDK |
| | THTCPPCPAPELLGGPSVFLFPPKPKDTLMISRTPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSREEM |
| | TKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| 40 | DVVMTQSPLSLPVTPGEPASISCRSSQSLLHSDGYNYLDWYLQKPG |
| | QSPQVLIYLGSNRASGVPDRFSGSGSGTDFTLKISRVEAEDVGVYY |
| | CMQALQTPFTFGPGTKVEIKRTVAAPSVFIFPPSDEQLKSGTASVV |
| | CLLNNFYPREAKVQWKVDNALQSGNSQESVTEQDSKDSTYSLSSTL |
| | TLSKADYEKHKVYACEVTHQGLSSPVTKSFNRGEC |

We claim:

1. An antibody that binds human LAIR1, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein
HCDR1 comprises SEQ ID NO: 1,
HCDR2 comprises SEQ ID NO: 2,
HCDR3 comprises SEQ ID NO: 3,
LCDR1 comprises SEQ ID NO: 4,
LCDR2 comprises SEQ ID NO: 5, and
LCDR3 comprises SEQ ID NO: 6.

2. The antibody of claim 1, wherein the VH comprises a sequence having at least 95% sequence identity to SEQ ID NO: 7, and the VL comprises a sequence having at least 95% sequence identity to SEQ ID NO: 8.

3. The antibody of claim 1, wherein the VH comprises SEQ ID NO: 7, and the VL comprises SEQ ID NO: 8.

4. The antibody of claim 1, wherein the antibody is a human antibody.

5. The antibody of claim 4, wherein the antibody has a human IgG2 or IgG4 isotype.

6. The antibody of claim 1, wherein the antibody comprises a heavy chain (HC) comprising SEQ ID NO: 9 and a light chain (LC) comprising SEQ ID NO: 10.

7. The antibody of claim 1, wherein the antibody comprises a HC comprising SEQ ID NO: 25 and a LC comprising SEQ ID NO: 10.

8. An antibody that binds human LAIR1, wherein the antibody comprises a VH and a VL, wherein the VH comprises HCDR1, HCDR2, and HCDR3, and the VL comprises LCDR1,
LCDR2, and LCDR3, wherein
HCDR1 comprises SEQ ID NO: 13,
HCDR2 comprises SEQ ID NO: 14,
HCDR3 comprises SEQ ID NO: 15,
LCDR1 comprises SEQ ID NO: 16,
LCDR2 comprises SEQ ID NO: 5, and
LCDR3 comprises SEQ ID NO: 18.

9. The antibody of claim 8, wherein the VH comprises a sequence having at least 95% sequence identity to SEQ ID NO: 19, and the VL comprises a sequence having at least 95% sequence identity to SEQ ID NO: 20.

10. The antibody of claim 8, wherein the VH comprises SEQ ID NO: 19 and the VL comprises SEQ ID NO: 20.

11. The antibody of claim 8, wherein the antibody is a human antibody.

12. The antibody of claim 11, wherein the antibody has a human IgG2 or IgG4 isotype.

13. The antibody of claim 8, wherein the antibody comprises a HC comprising SEQ ID NO: 21 and a LC comprising SEQ ID NO: 22.

14. The antibody of claim 8, wherein the antibody comprises a HC comprising SEQ ID NO: 27 and a LC comprising SEQ ID NO: 22.

15. The antibody of claim 1, wherein the antibody is an agonist of LAIR1.

16. The antibody of claim 1, wherein the antibody also binds cynomolgus monkey LAIR1.

17. The antibody of claim 8, wherein the antibody is an agonist of LAIR1.

18. The antibody of claim 8, wherein the antibody also binds cynomolgus monkey LAIR1.

19. A nucleic acid comprising a sequence encoding SEQ ID NO: 9, 25, 10, 21, 27 or 22.

20. A vector comprising the nucleic acid of claim 19.

21. The vector of claim 20, wherein the vector comprises a first nucleic acid sequence encoding SEQ ID NO: 9 or 25, and a second nucleic acid sequence encoding SEQ ID NO: 10.

22. The vector of claim 20, wherein the vector comprises a first nucleic acid sequence encoding SEQ ID NO: 21 or 27, and a second nucleic acid sequence encoding SEQ ID NO: 22.

23. A composition comprising a first vector comprising a nucleic acid sequence encoding SEQ ID NO: 9 or 25, and a second vector comprising a nucleic acid sequence encoding SEQ ID NO: 10.

24. A composition comprising a first vector comprising a nucleic acid sequence encoding SEQ ID NO: 21 or 27, and a second vector comprising a nucleic acid sequence encoding SEQ ID NO: 22.

25. A cell comprising the vector of claim 20.

26. A cell comprising a first vector comprising a nucleic acid sequence encoding SEQ ID NO: 9 or 25, and a second vector comprising a nucleic acid sequence encoding SEQ ID NO: 10.

27. A cell comprising a first vector comprising a nucleic acid sequence encoding SEQ ID NO: 21 or 27, and a second vector comprising a nucleic acid sequence encoding SEQ ID NO: 22.

28. The cell of claim 25, wherein the cell is a mammalian cell.

29. A process of producing an antibody comprising culturing the cell of claim 25 under conditions such that the antibody is expressed and recovering the expressed antibody from the culture medium.

30. An antibody produced by the process of claim 29.

31. A pharmaceutical composition comprising the antibody of claim 1, and a pharmaceutically acceptable excipient, diluent or carrier.

32. A pharmaceutical composition comprising the antibody of claim 8, and a pharmaceutically acceptable excipient, diluent or carrier.

33. A method of treating an autoimmune disease or a fibrotic disease in a subject in need thereof, comprising administering to the subject a therapeutically effective amount of the antibody of claim 1.

34. The method of claim 33, wherein the autoimmune disease or fibrotic disease is selected from rheumatoid arthritis, psoriasis, systemic lupus erythematosus, lupus nephritis pemphigus vulgaris, systemic sclerosis, idiopathic pulmonary fibrosis, scleroderma, ulcerative colitis, Crohn's disease, hidradenitis suppurativa, atopic dermatitis, multiple sclerosis, scleroderma-associated interstitial lung disease, IgG4 related disease or chronic fibrosing interstitial lung diseases.

35. A method of treating an autoimmune disease or a fibrotic disease in a subject in need thereof, comprising administering to the subject a therapeutically effective amount of the antibody of claim 8.

36. The method of claim 35, wherein the autoimmune disease or fibrotic disease is selected from rheumatoid arthritis, psoriasis, systemic lupus erythematosus, lupus nephritis pemphigus vulgaris, systemic sclerosis, idiopathic pulmonary fibrosis, scleroderma, ulcerative colitis, Crohn's disease, hidradenitis suppurativa, atopic dermatitis, multiple sclerosis, scleroderma-associated interstitial lung disease, IgG4 related disease or chronic fibrosing interstitial lung diseases.

37. The antibody of claim 1, wherein the antibody does not form a complex with LAIR1 ligand C1q.

38. The antibody of claim 1, wherein the antibody does not require full receptor occupancy (RO) to elicit agonism.

39. The antibody of claim 8, wherein the antibody does not form a complex with LAIR1 ligand C1q.

40. The antibody of claim 8, wherein the antibody does not require full receptor occupancy (RO) to elicit agonism.
